(19) United States
(12) Patent Application Publication
(10) Pub. No.: US 2023/0183810 A1
(43) Pub. Date: Jun. 15, 2023

(54) METHODS AND SYSTEMS FOR DETERMINATION OF AN EFFECTIVE THERAPEUTIC REGIMEN AND DRUG DISCOVERY

(71) Applicant: DNA-SEQ, Inc., San Diego, CA (US)

(72) Inventor: Janusz Sowadski, San Diego, CA (US)

(21) Appl. No.: 17/921,477

(22) PCT Filed: May 3, 2021
(86) PCT No.: PCT/US2021/030509

§ 371 Date: Oct. 26, 2022

Related U.S. Application Data
Application No. PCT/US2021/030509 is a continuation of application No. 16/866,281, filed on May 4, 2020.
(60) Provisional application No. 63/173,292, filed on Apr. 9, 2021.

(51) Int. Cl. C12Q 1/6886; G16B 15/00; G16B 30/00; G16B 40/00; G16B 50/00; G16C 20/30; G16B 15/30

(52) U.S. Cl. CPC C12Q 1/6886; G16B 15/00; G16B 15/30; G16B 30/00; G16B 40/00; G16B 50/00; G16C 20/30; C40B 30/04

(57) ABSTRACT

The present invention relates to the discovery of a method for identifying a treatment regimen for a patient diagnosed with cancer, predicting patient resistance to therapeutic agents and identifying new therapeutic agents, obtaining the specificity profile of a therapeutic agent, a method of designing a scaffold of a therapeutic agent directed against a drug-resistant target, drug scaffolds, and methods of uses thereof to identify drugs to treat diseases such as cancer. Specifically, the present invention relates to the use of an algorithm to identify a mutation in a kinase, determine if the mutation is an activation or resistance mutation and then to suggest an appropriate therapeutic regimen. The invention also relates to the use of a pattern matching algorithm and a crystal structure library to predict the functionality of a gene mutation, predict the specificity of small molecule kinase inhibitors and for the identification of new therapeutic agents.

### CROSS-REFERENCE TO RELATED APPLICATIONS

This application claims the benefit of priority under 35 U.S.C. §119(e) of U.S. Provisional Application Serial No. 63/173,292 filed Apr. 9, 2021, and under 35 U.S.C. §120 of U.S Application Serial No. 16/866,281, filed May 4, 2020, which is a Continuation-in-part of U.S. Application Serial No. 16/551,541 filed Aug. 26, 2019, which is a continuation application of U.S. Application Serial No. 15/346,671 filed Nov. 8, 2016, now issued as U.S. patent 10,392,669; which is a continuation-in-part application of U.S. Application Serial No. 14/606,918 filed Jan. 27, 2015, now issued as U.S. pat. 10,093,982, which claims the benefit under 35 USC § 119(e) to U.S. Application Serial No. 61/932,156 filed Jan. 27, 2014, now expired.. The disclosure of each of the prior applications is considered part of and is incorporated by reference in the disclosure of this application.

### BACKGROUND OF THE INVENTION

### Field of the Invention

The invention is directed generally to the prediction of the functionality associated with a gene mutation to identify appropriate therapeutic regimens based on known drugs and the development of novel therapeutics.

### Background Information

Cancer is one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Depending on the cancer type, patients typically have several treatment options available to them including chemotherapy, radiation and antibody-based drugs. Patients frequently develop resistance to one or more cancer treatments. Frequently this resistance is associated with a mutation in the tumor. There currently are no methods available to predict or monitor patients for the development of resistance to cancer treatments.

Complicating the treatment of cancer is the long timeline for the development of new chemotherapeutic agents. The current methodology of small molecule drug discovery is risky due to the lone and expensive development and clinical trial process that occurs prior to validation of the drug in patients. Additionally, the attrition rate for these drugs is high because determination of the drug candidate’s efficacy occurs late in the development process after massive expenditures have already occurred. The accumulated costs of the 4-6 years of pre-clinical and Phase 1 clinical trials are large and highly risky for the drug owner.

Thus, there is a need for more effective means for determining which patients will respond to specific cancer therapeutics, to predict which patients will develop resistance to cancer therapeutics and for incorporating such determinations into more effective treatment regimens for patients with anti-cancer therapies. Additionally, there is a need for better methods of quickly predicting which small molecules will be clinically beneficial prior to the need for expensive clinical trials.

Described herein is the use of a proprietary crystal structure library and a unique pattern matching algorithm to predict the functionality of a gene mutation, predict the specificity of a small molecule kinase inhibitor and to streamline drug development by the prediction of virtual molecules to inhibit kinases, for example by identifying previously unknown intermediate states of kinase catalytic cores resulting from activating cancer mutations. This predictive algorithm has been used to select appropriate therapeutic agents to target specific mutations as well as predict or monitor the development of resistance to therapeutic agents based in specific mutations. Further, the predictive algorithm methodology enables the rapid design of new drug candidates based on the specificity profile for the predicted functionality of a mutation.

### SUMMARY OF THE INVENTION

The present invention relates to the seminal discovery of a method for identifying a treatment regimen for a patient diagnosed with cancer, predicting patient resistance to therapeutic agents and identifying new therapeutic agents. Specifically, the present invention relates to the use of an algorithm to identify a mutation in a kinase, determine if the mutation is an activation or resistance mutation and then to suggest an appropriate therapeutic regimen. The invention also relates to the use of a pattern matching algorithm and a crystal structure library to predict the functionality of a gene mutation, predict the specificity of small molecule kinase inhibitors and for the identification of new therapeutic agents.

In one embodiment, the present invention provides a method for identifying a therapeutic regimen or predicting resistance to a therapeutic regimen for a patient with cancer comprising obtaining a biologic sample from the patient; identifying a at least one mutation in a gene sequence from the sample; using a pattern matching algorithm to determine if the at least one mutation is an activation mutation or a resistance mutation; and using the pattern matching algorithm and a crystal structure library to identify therapeutic agents to target the activating mutation or for which the patient is resistant; thereby identifying a therapeutic regimen or predicting resistance to a therapeutic regimen. In one aspect, the biological sample is blood, saliva, urine, bone marrow, serum, lymph, cerebrospinal fluid, sputum, stool, organ tissue or ejaculate sample. In an aspect, the at least one mutation is identified by sequence analysis. In one aspect, the at least one mutation is in the gene sequence of a receptor or a kinase. In another aspect, the receptor is an estrogen receptor. In a further aspect, the estrogen receptor is ESR1 or ESR2. In another aspect, the at least one mutation is in the catalytic domain of a kinase. In an additional aspect, the at least one mutation results in a novel kinase conformation. In a specific aspect, the at least one mutation is in the DFG domain. In a further aspect, the crystal structure library comprises a protein crystal structure database and a therapeutic agent crystal structure database. In an additional aspect, the algorithm is subjected to machine learning. In one aspect, the at least one mutation comprises an activation mutation or a resistance mutation. In another aspect, the at least one mutation comprises a mutation in a kinase or a receptor. In certain aspects, the receptor is an estrogen receptor 1 (ESR1) or an estrogen receptor 2 (ESR2). In another aspect, the therapeutic regimen comprises a kinase inhibitor and/or a chemotherapeutic agent. In another aspect, the method further comprises using a three-dimensional template to identify therapeutic agents.

In one embodiment, the present invention relates to a method of determining risk for developing resistance or the development of resistance to a therapeutic regimen in an ER+ breast cancer patient comprising obtaining a biological sample and a tumor sample from the patient; contacting each sample with a probe that binds to a sequence in a gene associated with kinase phosphorylation; and comparing the binding of the probe in the biological sample with the binding of the probe in the tumor sample wherein binding of the probe with the biological sample but not the tumor sample is indicative of a tumor that is at risk for developing resistance to a therapeutic regimen. In one aspect, the sample is obtained from the patient following a course of therapy and wherein the course of therapy is ongoing for at least about 1 month to 6 months at the time the sample is obtained. In another aspect, the sample is obtained at intervals throughout the course of therapy. In one aspect, the subject is a human. In a further aspect, the biological sample is blood, saliva, urine, bone marrow, serum, lymph, cerebrospinal fluid, sputum, stool, organ tissue or ejaculate sample. In an additional aspect, the probe detects a mutation in the gene sequence. In a specific aspect, the mutation is a point mutation. In another aspect, the biological sample is a tumor sample and specifically, the tumor sample is a liquid biopsy or a sample of circulating tumor cells (CTCs).

In another aspect, the probe detects a deletion in the gene sequence. In one aspect, the deletion is about 2 to 12 amino acids. In a further aspect, the probe detects a deletion and a single point mutation in the gene sequence. In one aspect probe is at least about 1000 nucleotides, from about 300 to 500 nucleotides or at least about 150 nucleotides for more than one region of the gene sequence. In further aspect, the gene sequence is an ESR receptor gene sequence. In a specific aspect, the ESR receptor is ESR1 or ESR2. In certain aspects, the ESR1 receptor has a point mutation at Y537, E380, L536, and/or D538. In specific aspects the ESR1 mutation is Y537S, Y537A, Y537E or Y537K. In another aspect, the ESR2 receptor has a point mutation at V497 and specifically, the mutation is V497M.

In a further aspect, the therapeutic regimen is treatment with an aromatase inhibitor. In a specific aspect, the therapeutic regimen is treatment with a tamoxifen, Raloxifene and/or a competitor of estrogen in its ER binding site.

In another aspect, the method further includes predicting a second form of therapy. In certain aspects, the second form of therapy is provided to the patient prior to completion of a therapeutic regimen with a first form of therapy. In another aspect, the first form of therapy is an aromatase inhibitor and the second form of therapy is a non-aromatase inhibitor chemotherapeutic drug. In an additional aspect, the non-aromatase inhibitor chemotherapeutic drug may be antimetabolites, such as methotrexate, DNA cross-linking agents, such as cisplatin/carboplatin; alkylating agents, such as canbusil; topoisomerase I inhibitors such as dactinomycin; microtubule inhibitors such as TAXOL™ (paclitaxel), a vinca alkaloid, mitomycin-type antibiotic, bleomycin-type antibiotic, antifolate, colchicine, demecolcine, etoposide, taxane, anthracycline antibiotic, doxorubicin, daunorubicin, caminomycin, epirubicin, idarubicin, mitoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methotrexate, lomustine, thioguanine, asparaginase, vinblastine, vindesine, tamoxifen, or mechlorethamine, antibodies such as trastuzumab; bevacizumab, OSI-774, Vitaxin; alkaloids, including, microtubule inhibitors (e.g., Vincristine, Vinblastine, and Vindesine, etc.), microtubule stabilizers (e.g., Paclitaxel (TAXOL™ (paclitaxel), and Docetaxel, Taxotere, etc.), and chromatin function inhibitors, including, topoisomerase inhibitors, such as, epipodophyllotoxins (e.g., Etoposide (VP-16), and Teniposide (VM-26), etc.), agents that target topoisomerase I (e.g., Camptothecin and Isirinotecan (CPT-11), etc.); covalent DNA-binding agents (alkylating agents), including, nitrogen mustards (e.g., Mechlorethamine, Chlorambucil, Cyclophosphamide, Ifosphamide, and Busulfan (MYLERAN™), etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); noncovalent DNA-binding agents (antitumor antibiotics), including, nucleic acid inhibitors (e.g., Dactinomycin (Actinomycin D)), anthracyclines (e.g., Daunorubicin (Daunomycin, and Cerubidine), Doxorubicin (Adriamycin), and Idarubicin (Idamycin)), anthracenediones (e.g., anthracycline analogues, such as, (Mitoxantrone)), bleomycins (Blenoxane), etc., and plicamycin (Mithramycin); antimetabolites, including, antifolates (e.g., Methotrexate, Folex, and Mexate), purine antimetabolites (e.g., 6-Mercaptopurine (6-MP, Purinethol), 6-Thioguanine (6-TG), Azathioprine, Acyclovir, Ganciclovir, Chlorodeoxyadenosine, 2-Chlorodeoxyadenosine (CdA), and 2′-Deoxycoformycin (Pentostatin), etc.), pyrimidine antagonists (e.g., fluoropyrimidines (e.g., 5-fluorouracil (Adrucil), 5-fluorodeoxyuridine (FdUrd) (Floxuridine)) etc.), and cytosine arabinosides (e.g., Cytosar (ara-C) and Fludarabine); enzymes, including, L-asparaginase; hormones, including, glucocorticoids, such as, antiestrogens (e.g., Tamoxifen, etc.), nonsteroidal antiandrogens (e.g., Flutamide); platinum compounds (e.g., Cisplatin and Carboplatin); monoclonal antibodies conjugated with anticancer drugs, toxins, and/or radionuclides, etc.; biological response modifiers (e.g., interferons (e.g., IFN-alpha.) and interleukins (e.g., IL-2).

In one aspect, the determination is performed on a computer. In another aspect, the gene sequence is in a database. In a certain aspect, the database contains sequences for the catalytic cores of protein kinases.

In a further embodiment, the present invention provides a method for identifying a drug candidate comprising identifying a mutation for resistance to a first drug by genomic and/or three-dimensional crystallographic analysis; and determining a second drug based on the mutation for resistance due to the first drug, by searching a crystal structure library database to identify a scaffold for a drug candidate as the second drug, thereby identifying a drug candidate. In one aspect, a pattern matching algorithm is used to search the crystal structure library.

In another embodiment, the present invention provides a method for predicting the specificity profile of a therapeutic agent comprising obtaining the crystal structure of the therapeutic agent; and using a pattern matching algorithm to identify targets of the therapeutic agent using a crystal structure library, thereby, predicting the specificity profile of a therapeutic agent. In one aspect, the crystal structure library comprises a protein crystal structure database. In another aspect, the protein crystal structure database comprises the crystal structure of kinases and receptors. In an aspect, the therapeutic agent is a kinase inhibitor. In one aspect, the kinase inhibitor is Afatinib, Axitinib, Bevacizumab, Bosutinib, Cetuximab, Crizotinib, Dasatinib, Erlotinib, Fostamatinib, Gefitinib, Ibrutinib, Imatinib, Lapatinib, Lenvatinib, Masitinib, Mubritinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib or Vemurafenib or a combination thereof. In another aspect, the therapeutic agent is a chemotherapeutic agent. In an additional aspect, the target is a kinase or a receptor. In one aspect, the target is a mutation in a gene sequence. In a further aspect, the gene mutation is in a kinase or a receptor. In certain aspects, the target is the catalytic domain of a kinase. In a specific aspect, the target is the DFG domain. In one aspect, the receptor is an estrogen receptor. In an additional aspect, the specificity profile is used in the selection of a treatment regimen for a patient in need thereof.

In a further embodiment, the present invention provides a method of treating a patient in need thereof comprising obtaining a biologic sample; identifying at least one mutation in a gene from the biologic sample; using a pattern matching algorithm and a crystal structure library to identify at least one therapeutic agent to target the at least one mutation; and administering the identified therapeutic agent to the patient, thereby treating the patient. In one aspect, the patient is diagnosed with cancer. In another aspect, at least 2 gene mutations are identified. In certain aspects, 2, 3, 4, 5, 6, 7, 8, 9, or 10 gene mutations are identified. In a further aspect, the gene mutations are identified by sequence analysis. In an aspect, the crystal structure library comprises the crystal structure of kinases, receptors and ligands. In one aspect, the target is a kinase or a receptor. In an additional aspect, more than one therapeutic agent is selected for the treatment regimen. In a further aspect the at least one chemotherapeutic agent. In certain aspects, one chemotherapeutic agent is a kinase inhibitor. In another aspect, the method further comprises using a three-dimensional template to identify at least one therapeutic agent.

In a further embodiment, the invention provides for a method of determining a disease state in a subject comprising obtaining a biological sample and a sample suspected of containing diseased cells from the subject; contacting each sample with a probe that binds to a sequence in a gene associated with kinase phosphorylation; and comparing the binding of the probe in the biological sample with the binding of the probe in the diseased cell sample wherein binding of the probe with the biological sample but not the diseased cell sample is indicative of a disease state or risk for developing a disease state in a subject. In one aspect, the disease state may be cancer, autoimmunity, infectious disease, and genetic disease. In an aspect, the method further comprises identifying a disease therapy, monitoring treatment of a disease state, determining a therapeutic response, identifying molecular targets for pharmacological intervention, and making determinations such as prognosis, disease progression, response to particular drugs and to stratify patient risk. In an additional aspect, the method further comprises determining a proliferation index, metastatic spread, genotype, phenotype, disease diagnosis, drug susceptibility, drug resistance, subject status and treatment regimen. In another aspect, the biological sample is blood, saliva, urine, bone marrow, serum, lymph, cerebrospinal fluid, sputum, stool, organ tissue, ejaculate sample, an organ sample, a tissue sample, an alimentary/gastrointestinal tract tissue sample, a liver sample, a skin sample, a lymph node sample, a kidney sample, a lung sample, a muscle sample, a bone sample, or a brain sample, a stomach sample, a small intestine sample, a colon sample, a rectal sample, or a combination thereof. In a further, aspect, the cancer is selected from an alimentary/gastrointestinal tract cancer, a liver cancer, a skin cancer, a breast cancer, an ovarian cancer, a prostate cancer, a lymphoma, a leukemia, a kidney cancer, a lung cancer, an esophageal cancer, a muscle cancer, a bone cancer, or a brain cancer. In certain aspects, the cancer is breast cancer, and the breast cancer is ER+ breast cancer. In an aspect, the drug is a chemotherapeutic drug, an antibiotic, or an anti-inflammatory drug. In another aspect, the subject is a mammal and specifically, the human subject is a human.

In an additional embodiment, the present invention provides for a system for automated determination of an effective protein kinase inhibitor drug for a patient in need thereof comprising an input operable to receive patient sequence data for a protein kinase suspected of being associated with a disease state; a processor configured to apply the received sequence data to a first database comprising three-dimensional models of crystal structures of protein kinases, the processor configured to provide a display aligning a native protein kinase with the patient’s protein kinase sequence, thereby identifying a region in the three-dimensional crystal structure of the kinase where the patient’s kinase differs from the native kinase. In one aspect, the method further comprises a processor for input from a second database, wherein the second database comprises a plurality of protein kinase inhibitor drugs, thereby allowing stratification of one or more drug treatment options in a report based on the output status of the patient sequence data and the protein kinase inhibitor drugs. In an additional aspect, the patient is a cancer patient. In another aspect, the kinase is a tyrosine kinase.

In one embodiment, the present invention provides for a method of determining a therapeutic regimen for a patient comprising utilizing the system described above to determine one or more drugs for which the patient will be responsive and administering the one or more drugs to the patient based on the stratifying. In another aspect, the stratifying further comprises ranking one or more drug treatment options with a higher likelihood of efficacy or with a lower likelihood of efficacy. In another aspect, the stratifying further comprises ranking one or more drug treatment options with a higher likelihood of developing drug resistance of a lower likelihood of developing drug resistance. In a further aspect, the stratifying is indicated by color coding the listed drug treatment options on the report based on a rank of a predicted efficacy or resistance of the drug treatment options. In one aspect, the annotating comprises using information from a commercial database. In a further aspect, the annotating comprises providing a link to information on a clinical trial for a drug treatment option in the report. In one aspect, the annotating comprises adding information to the report selected from the group consisting of one or more drug treatment options, scientific information regarding one or more drug treatment options, one or more links to scientific information regarding one or more drug treatment options, one or more links to citations for scientific information regarding one or more drug treatment options, and clinical trial information regarding one or more drug treatment options.

In an additional embodiment, the present invention provides for a system for automated determination of an effective protein kinase inhibitor drug for a patient in need thereof comprising a database; and a processor circuit in communication with the database, the processor circuit configured to receive patient sequence data for a protein kinase suspected of being associated with a disease state; identify data indicative of a disease state within the database; store the data indicative of the disease state in the database; organize the data indicative of the disease state based on disease state; analyze the data indicative of the disease state to generate a treatment option based on the disease state and protein kinase inhibitor drug; and cause the treatment option and the organized data to be displayed.

In a further embodiment, the present invention provides for a method of determining a second course of therapy for a subject having developed resistance for a first course of therapy comprising identifying a mutation for resistance to the first course of therapy by genomic and/or three-dimensional crystallographic analysis; and determining a drug for the second course of therapy based on a search of a database of existing drugs, thereby identifying the second course of therapy.

In one embodiment, the present invention provides for a method of determining a second course of therapy for a subject having developed resistance for a first course of therapy comprising identifying a mutation for resistance to the first course of therapy by genomic and/or three-dimensional crystallographic analysis; and determining a drug for the second course of therapy based on a search of a crystal structure library database to identify a scaffold for a drug candidate as the second course of therapy, thereby identifying the second course of therapy. In an aspect, the determining step uses a quantum computer.

In an additional embodiment, the present invention provides for a method for identifying a drug candidate comprising: identifying a mutation for resistance to a first course of therapy by genomic and/or three-dimensional crystallographic analysis; and determining a drug for the second course of therapy based on a search of a database of existing drugs and the genomic and/or three-dimensional crystallographic analysis, thereby identifying a drug candidate.

In yet another embodiment, the invention provides a method for obtaining the specificity profile of a therapeutic agent including obtaining the crystal structure of the therapeutic agent; identifying a DFG phosphate conformation on a target of the therapeutic agent using a algorithmic phosphate detector; and obtaining the specificity profile of the therapeutic agent using the conformation of the phosphate on the target and a pattern matching algorithm with a crystal structure library, thereby obtaining the specificity profile of a therapeutic agent. In one aspect, the phosphate is located on an activation loop of the target. In various aspects, the phosphate is a DFG IN conformation, a DFG OUT conformation, or a DFG INTERMEDIATE conformation. In another aspect, the conformation of the phosphate indicates if the target is in an active state or in an inactive state. In one aspect, a DFG IN conformation of the phosphate indicates an active state of the target, a DFG OUT conformation indicates an inactive state of the target, and a DFG INTERMEDIATE conformation indicates an active state of the target. In many aspects, the target is a kinase. In one aspect, the therapeutic agent is a kinase inhibitor. In some aspects, the therapeutic agent is a chemotherapeutic agent. In various aspects, the chemotherapeutic agent is selected from the group consisting of dasatinib, imatinib, nilotinib, bosutinib, regorafenib, sorafenib, ponatinib, sunitinib, vermurafenib, vandetanib, ibrutinib, abemaciclib, ribociclib, palbociclib, axitinib, crizotinib, gilteritinib, erlotinib, midstaurin, ruxolitinib, brigatinib and osimeritinib. In one aspect, a mutation in a gene encoding the target results in a change in the conformation of the phosphate on the target. In some aspects, the mutation induces a lack of detection of a phosphate on DFG INTERMEDIATE conformation on the target. In other aspects, the mutation is an activating mutation or a drug resistance mutation. In one aspect, the crystal structure library includes a kinase crystal structure database, a receptor crystal structure database, and/or a therapeutic agent crystal structure database. In some aspects, the therapeutic agent is a drug for the treatment of cancer.

In another embodiment, the invention provides a method of designing a scaffold of a therapeutic agent directed against a drug-resistant target including creating a three dimensional fishing net of the distances in the DFG phosphate conformation (3D surface net) of the drug resistant target using a algorithmic phosphate detector; screening a library of small fragment to capture small fragment that specifically binds to the 3D surface net of the target, thereby identifying a scaffold structure of the therapeutic agent; and using a fragmentation algorithm to deconstruct the resistant drug structure and construct the scaffold of a therapeutic agent from the surface net structure and the captures small fragment. In one aspect, the target is a kinase, and the therapeutic agent is a kinase inhibitor. In another aspect, a mutation in a gene encoding the target results in a change in the conformation of the phosphate on the target. In some aspects, the mutation induces a phosphate to be in a DFG INTERMEDIATE conformation. In various aspects, the drug-resistant target is in a DFG INTERMEDIATE conformation, and the 3D surface net is a 3D INTERMEDIATE surface net (3D INTER net). In one aspect, creating the three-dimensional fishing net includes excluding regions of high frequency of drug resistance mutation of the kinase. In some aspects, regions of high frequency of drug resistance mutation of the kinase comprises HVR regions 1-7. In another aspect, creating the three-dimensional fishing net includes minimizing the chemical scaffold using the adenosyl ring of adenosine triphosphate (ATP). In one aspect, screening the library includes limiting the molecular weight of the therapeutic agent to about 400 kDa. In another aspect, the method further includes identifying analogs of the scaffold to identify the therapeutic agent. In some aspects, the scaffold of the therapeutic agent is defined by two derivation constituents. In many aspects, the two derivation constituents are left open to generate a combinatorial array of analogs. In some aspects, a first derivation constituent (R1) improves stability of the scaffold and minimize toxicity of the therapeutic agent. In other aspects, a second derivation constituent (R2) improves conformational specificity of the scaffold and maximize affinity of the therapeutic agent to the scaffold. In one aspect, the kinase is ABL1 mutant T315I. In one aspect, the scaffold of the therapeutic agent is selected from

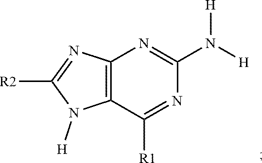

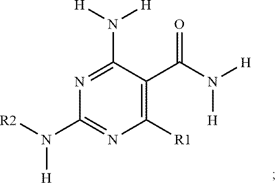

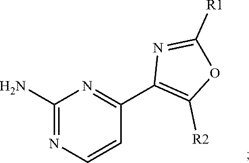

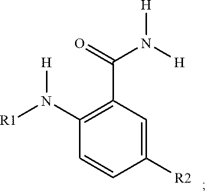

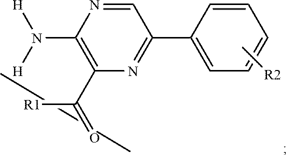

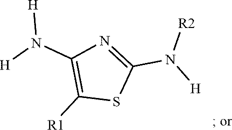

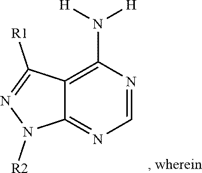

R1 and R2 are two derivation constituents.

In one embodiment, the invention provides a method of treating cancer in a subject including administering to the subject a therapeutically effective amount of a therapeutic agent, wherein the cancer is characterized by a mutation in an ABL1 gene, and wherein the therapeutic agent is selected from

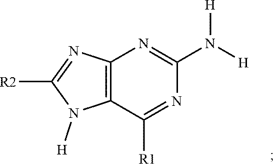

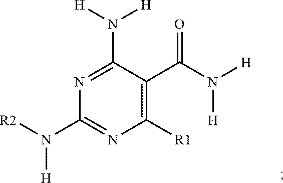

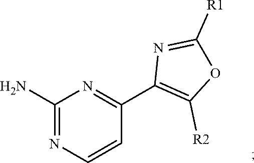

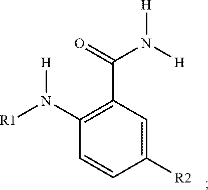

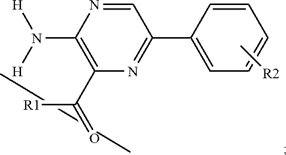

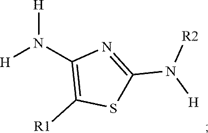

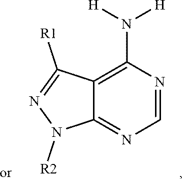

wherein R1 and R2 are two derivation constituents, thereby treating cancer in the subject. In one aspect, the cancer is resistant to one or more tyrosine kinase inhibitors (TKIs).in some aspects, the one or more TKIs are selected from the group consisting of imatinib, nilotinib, dasatinib, bosutinib, ibrutinib and ponatinib. In other aspects, the ABL1 mutation is T315I.

In another embodiment, the present invention provides a method of treating leukemia in a subject including administering to the subject a therapeutically effective amount of a cyclin dependent kinase (CDK) inhibitor, wherein the CDK inhibitor is NU6027, thereby treating leukemia in the subject.

In one aspect, the leukemia is resistant to one or more tyrosine kinase inhibitors (TKIs). In some aspects, the one or more TKIs are selected from the group consisting of imatinib, nilotinib, dasatinib, bosutinib, ibrutinib and ponatinib. In another aspect, the leukemia is characterized by a mutation in an ABL1 gene. In some aspects, the ABL1 mutation is T315I. In one aspect, the subject has previously been treated with imatinib, nilotinib, dasatinib, ibrutinib, ponatinub, or a combination thereof. In another aspect, the leukemia is selected from the group consisting of acute lymphoblastic leukemia (ALL), acute myeloid leukemia (AML), blastic plasmacytoid dendritic cell neoplasm (BPDCN), chronic lymphocytic leukemia (CLL), chronic myelogenous leukemia (CML), hairy cell leukemia, mast cell leukemia and meningeal leukemia. In some aspects, the leukemia is CML. In one aspect, NU6027 binds to a mutated ABL1 kinase domain in either a phosphorylated or unphosphorylated conformation. In some aspects, NU6027 binds to a mutated ABL1 kinase domain with a K_{D} that is at least 100 times greater that the K_{D} of imatinib, nilotinib, dasatinib, bosutinib, ibrutinib or ponatinib. In another embodiment, the invention provides a method of treating a drug-resistant chronic myelogenous leukemia (CML) in a subject comprising: administering to the subject a therapeutically effective amount of NU6027, thereby treating the drug-resistant CML in the subject. In one aspect, the CML is resistant to imatinib, nilotinib, dasatinib, bosutinib and/or ponatinib.

### BRIEF DESCRIPTION OF THE DRAWINGS

FIGS. 1A-1E show the use of the 3D pattern matching algorithm for the selection of therapeutic agents to target a specific kinase mutation. FIG. 1A. The unique 3D pattern for fast sorting of scaffolds. FIG. 1B. The unique 3D hydrogen bond pattern with the scaffolds, including imatinib. FIG. 1C. The unique 3D hydrogen bond pattern in the target for binding the scaffold. FIG. 1D. The unique 3D pattern (subgraphs) of the scaffold to identify target specific binding pockets. FIG. 1E. The unique 3D pattern of the target which allow the algorithm to fast walk through the polypeptide chain of the target.

FIGS. 2A-2C show the prediction of kinase domain conformation of a mutation identified from a patient. FIG. 2A. The identification of the phosphorylation sites on the target, including the activation loop. FIG. 2B. The selection of a unique 3D pattern within the DFG motif of the target to identify intermediate DFG conformations. FIG. 2C. The unique 3D pattern of the hydrophobic core of the target to identify a common drug resistance mutation.

FIGS. 3A-3F show the prediction of a specificity profile of a small molecule kinase inhibitor. FIG. 3A. Identification of the three-dimensional network of selected constant (conserved) amino acids in the target. FIG. 3B. Identification of the three-dimensional network of the variable (non-conserved) residues of the target. FIG. 3C. Selection of unique 3D pattern combinations for the prediction of the specificity profile for dasatinib. FIG. 3D. The unique combination of 3D patterns defining specific chemical interactions of the target and inhibitor to predict low and high affinities of nilotinib. FIG. 3E. The 3D structure of masitinib fitted onto the crystal structure of imatinib. FIG. 3F. Experimental versus computation specificity profiles for masitinib.

FIGS. 4A-4D show the determination that a kinase mutation is activating. FIG. 4A. Evidence of the D816 mutation in KIT, building and regularizing the model of the mutant. FIG. 4B. Pattern matching of the model to determine DFG in conformation. FIG. 4C. Pattern matching of the model to determine DFG out conformation. FIG. 4D. Pattern matching of the model to determine DFG intermediate conformation.

FIG. 5 shows superimposition of C-KIT structures from DNA-SEQ library ZZ00617 (PDB ID 1KPG) and ZZ00618 (PDB ID 1T46). ZZ00617 has DFG in conformation IN and ZZ00618 has DFG in conformation OUTL. Visualized from the top of the kinase. The transparency of the electrostatic surface makes it possible to observe structure details, like STA1, that coincide in both structures, DFG OUTL and DFG IN, the two different conformation of activation loops, the mutant position T670 and conserved position E640.

FIG. 6 shows the same structure as FIG. 5 from another perspective. The same details are visualized to identify axis y passing through T670, axis x passing through E640, and axis z passing through ADP exit trajectory.

FIG. 7 is a schematic diagram of the bi-lobal homologous catalytic core of a protein kinase, with a two-fold roto-translation axis, and the DGF motif outlined (arrow). The circle indicates the activation loop that is detailed in FIG. 8.

FIG. 8 shows C (alpha) coordinates of the first crystal structure of kinase (PKA pdb id: 1ATP) encompassing DFG motif and activation loop. This part of the catalytic core is firmly embedded in the most conserved part of the core, yet by itself it is highly diverse, except the canonical DFG motif. It is the critical highly flexible part that transmits the activation process (phosphorylation) into enzymatic action (PKA is always active as it is activated by release of regulatory subunit upon cAMP binding). The activation mechanism is diverse among kinases; for example, auto-phosphorylation requires release of inhibitory domain to allow ATP entry. Hence this type of mechanism requires binding of the signaling molecules (hormones, CA (+2) etc.). The STA1 (from a static) position usually has low temperature factors. This is the first hydrophobic residue in the “pivot 1 range”. Following that, the residue HV7 is highly variable and forms a critical part of “pivot1”. Certain kinases “switch” from the IN to the OUT DFG conformation at that point. Following HV7 is the DFG motif as reported in the previous section it is the highly dynamic motif which binds two metal sites (specifically D). Following DFG motif is the residue P1 (pivot 1). This is the critical highly diverse residue among kinases, and it is very rich in cancer “activation” mutations. Based on our pattern matching analysis this residue is responsible for the high conformational diversity of the DFG motif, as previously mentioned. The residues, 1 through 14, represent the highly diverse activation loop. Here the kinase subfamily is highly differentiated and phosphorylation (or auto phosphorylation) can occur at any residue, depending on the kinase or kinase subfamily. P2 (“pivot 2”) is a series of three residues in a short alpha helix conformation. At P2 many kinases undergo an IN/OUT conformation change which alters the surrounding structural microenvironment.

FIGS. 9A-9D show the design and training results for the machine learning algorithm in defining DFG conformations. FIG. 9A illustrates DFG explicit with backbone and active site AS1 and AS2. Geometrical descriptor explicit to teach the algorithm with angles and orientation. FIG. 9B is a representation of tortuosity for DFG and activation loop, the tortuosity is described from STA1 (HVR7) through DFG and till END (last amino acid in activation loop). FIG. 9C is an explicit representation of the four class of DFG conformation identified by the algorithm: DFG IN, DFG OUT, DFG OUTL, and DFG INTER. FIG. 9D illustrates that after the optimization of the training algorithm and its calibration the algorithm has being run against the full DNA SEQ library identifying the population of the four DFG conformations as the histograms show. Specifically, DFG IN, DFG INTER, DFG OUT, DFG OUTL.

FIG. 10 shows the three-dimensional structures of ABL1 in the three conformations identified by algorithm from left DFG IN, center DFG INTER, right DFG IN. ABL1 structure on the left is bound with dasatinib and shows two water molecules in the ATP binding site, ABL1 structure in the center is bound to ATP analog and shows ten water molecules in the site, and the ABL1 structure on the right is bound to Imatinib and shows two water molecules in the site. The population of the specific conformation in the DNA SEQ crystal library is below each structure. Population is calculated with a trained DNA SEQ algorithm.

FIGS. 11A-11D. FIG. 11A show the details of crystal structure ZZ00417 for ABL1 in conformation DFG INTER, superposed to Imatinib DFG-OUT and dasatinib DFG-IN binding site in the ATP binding pocket. Spheres represent the HVR 1 to 7 residues and the axis of rotation passing through HVR4 and HVR7. FIG. 11B is a graph illustrating statistics representing the distances (in A) between C alpha HVR4 and C alpha HVR7.The error bars for DFG IN (ABL1) and DFG OUT (ABL1) did not overlap the DFG INTER (ABL1 and CDK2) error bars. DFG INTER for the ABL1 has only a single data which is supported by the chart on the right side for the DFG INTER of CDK2 which has a dataset of thirty-two structures. FIG. 11C illustrates the localization of mutations in the HVR4 location. FIG. 11D shows the distribution of ABL1 imatinib resistance mutation from 1127 patients calculated as the number of cancer patients treated by imatinib per each specific drug resistance mutation. Distribution shows three major regions populated by resistance mutation in the ABL1 catalytic domain, the highest single pick of mutations in region two is only represented by HVR4 location (T315 in ABL1).

FIGS. 12A-12D illustrate the algorithm DFG classifications, Fishing Net and Virtual sorting description. FIG. 12A is a pie chart of DFG classification after trained algorithm analyzed full DNA SEQ crystal library. On a total of 1616 structures analyzed, the algorithm found: 933 DFG IN, 79 OUT, 136 OUTL, 228 DFG INTER with 1 Å shortening GAP in HVR4-HVR7 axis, 240 DFG INTER without the 1 Å shortening GAP in HVR4-HVR7 axis. FIG. 12B is a schematic representation of the fishing net characteristic. The fishing net prohibits the protruding of any primary DFG INTER binding scaffold inside the HVR7 three-dimensional network. FIG. 12C illustrates the superposition of ATP, ADP and analogs from DNA SEQ library showing the natural way to bind without protruding through HVR7 network. All atoms are out or at the threshold of the network with a specific alignment with the HVR4-HVR7 axis. FIG. 12D is a generic and schematic representation of DNA SEQ selected scaffold showing the possible way to functionalize to increase affinity for DFG INTER and other ADME, QSAR and solubility. The R1 substitution is define by cancer and a specific conformation of an amino acid. The R2 which aim to solution site is the free area to improve drug like characteristic for the selected scaffold. Both R1 and R2 are suitable to generate combinatorial chemistry for each scaffold.

FIG. 13 illustrates the chemical structures of J, A, N, U, S, Z and M scaffolds using the virtual sort and the fishing net.

FIG. 14 is a vector map illustrating the construct used for the expression of the kinase domain (residues 229-499) of a mutant ABL1 protein including a T315I mutation.

FIG. 15 is a blot illustrating the efficient removal of the His tag attachment by the TEV protease.

FIG. 16 shows a Coomassie stained quantitative gel of partially purified and high purity unphosphorylated mutant ABL1_T315I kinase domain.

FIGS. 17A-17B illustrate the densitometry analysis of mutant ABL1_T315I kinase domain. FIG. 17A illustrates the densitometry analysis of 1 µg partially purified mutant ABL1_T315I kinase domain. FIG. 17B illustrates the densitometry analysis of 1 µg high purity mutant ABL1_T315I kinase domain.

FIGS. 18A-18B illustrate melt curves of tag removed mutant ABL1_T315I kinase domain. FIG. 18A illustrates melt curves of tag removed mutant ABL1_T315I kinase domain from TSA. FIG. 18B illustrates melt curves of tag removed mutant ABL1_T315I kinase domain from first derivative.

FIGS. 19A-19C illustrate the analysis of the phosphorylation status of mutant ABL1_T315I kinase domain by liquid chromatography-mass spectrometry (LC-MS). FIG. 19A illustrates the LC profile. FIG. 19B illustrates the LC profile with an electrospray ionization. FIG. 19C illustrates the deconvoluted spectra.

FIG. 20 shows an immunoblot of 10x-His-tev-ABL1(T315I)(299-449) illustrating the autophosphorylation ability of the kinase domain.

FIGS. 21A-21D illustrate sensorgrams of A-0001. FIG. 21A illustrates the sensorgram of A-0001 binding to the unphosphorylated form of ABL1(T315I)(299-449). FIG. 21B illustrates the sensorgram of A-0001 binding to the unphosphorylated form of ABL1(T315I)(299-449) fitted using an equilibrium steady state model. FIG. 21C illustrates the sensorgram of A-0001 binding to the phosphorylated form of ABL1(T315I)(299-449). FIG. 21D illustrates the sensorgram of A-0001 binding to the phosphorylated form of ABL1(T315I)(299-449) fitted using an equilibrium steady state model.

FIGS. 22A-22D illustrate sensorgrams of J-0024. FIG. 22A illustrates the sensorgram of J-0024 binding to the unphosphorylated form of ABL1(T315I)(299-449). FIG. 22B illustrates the sensorgram of J-0024 binding to the unphosphorylated form of ABL1(T315I)(299-449) fitted using an equilibrium steady state model. FIG. 22C illustrates the sensorgram of J-0024 binding to the phosphorylated form of ABL1(T315I)(299-449). FIG. 22D illustrates the sensorgram of J-0024 binding to the phosphorylated form of ABL1(T315I)(299-449) fitted using an equilibrium steady state model.

FIGS. 23A-23D illustrate sensorgrams of imatinib. FIG. 23A illustrates the sensorgram of imatinib binding to the unphosphorylated form of ABL1(T315I)(299-449). FIG. 23B illustrates the sensorgram of imatinib binding to the unphosphorylated form of ABL1(T315I)(299-449) fitted using an equilibrium steady state model. FIG. 23C illustrates the sensorgram of imatinib binding to the phosphorylated form of ABL1(T315I)(299-449). FIG. 23D illustrates the sensorgram of imatinib binding to the phosphorylated form of ABL1(T315I)(299-449) fitted using an equilibrium steady state model.

### DETAILED DESCRIPTION OF THE INVENTION

The present invention relates to the seminal discovery of a method for identifying a treatment regimen for a patient diagnosed with cancer, predicting patient resistance to therapeutic agents and identifying new therapeutic agents. Specifically, the present invention relates to the use of an algorithm to identify a mutation in a kinase, determine if the mutation is an activation or resistance mutation and then to suggest an appropriate therapeutic regimen. The invention also relates to the use of a pattern matching algorithm and a crystal structure library to predict the functionality of a gene mutation, predict the specificity of small molecule kinase inhibitors and for the identification of new therapeutic agents.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. The definitions set forth below are for understanding of the disclosure but shall in no way be considered to supplant the understanding of the terms held by those of ordinary skill in the art.

In one embodiment, the present invention provides a method for obtaining the specificity profile of a therapeutic agent including obtaining the crystal structure of the therapeutic agent; identifying a DFG phosphate conformation on a target of the therapeutic agent using a algorithmic phosphate detector; and obtaining the specificity profile of the therapeutic agent using the conformation of the phosphate on the target and a pattern matching algorithm with a crystal structure library, thereby obtaining the specificity profile of a therapeutic agent. In one aspect, the phosphate is located on an activation loop of the target. In many aspects, the target is a kinase.

A kinase is a type of enzyme that catalyzes the transfer of phosphate groups from high-energy, phosphate-donating molecules to specific substrates. Kinases are critical in metabolism, cell signaling, protein regulation, cellular transport, secretory processes, and countless other cellular pathways. Kinases mediate the transfer of a phosphate moiety from a high energy molecule (such as ATP) to their substrate molecule. Kinases are needed to stabilize this reaction because the phosphor-anhydride bond contains a high level of energy. Kinases properly orient their substrate and the phosphoryl group within in their active sites, which increases the rate of the reaction. Additionally, they commonly use positively charged amino acid residues, which electrostatically stabilize the transition state by interacting with the negatively charged phosphate groups. Alternatively, some kinases utilize bound metal cofactors in their active sites to coordinate the phosphate groups.

Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme.

The crystal structure 1ATP contains the mouse PKA catalytic (C) subunit, inhibitor protein PKI, the ATP analog ANP (CPK wireframe), and two manganese ions. In addition to the protein kinase catalytic domain (residues 43-297), the C subunit contains amino-terminal (residues 1-43) and carboxy-terminal (residues 298-350) sequences. The protein kinase fold of catalytic domains of eukaryotic protein kinases comprises a small lobe and a large lobe with a catalytic cleft, marked by the bound ANP molecule, is located between them. The small lobe binds ATP and the large lobe binds the protein substrate, modeled here by the inhibitor peptide PKI. PKI has an alanine substituted for the serine in the phosphorylation motif RRxS, and thus is unable to be phosphorylated.

The catalytic domain (i.e., protein kinase domain) is comprised of twelve subdomains:

Subdomain I contains two beta strands connected by the glycine-rich ATP-binding loop with the motif GxGxxG shown.

Subdomain II contains an invariant lysine that interacts with the phosphates of ATP.

Subdomain III is an alpha helix (helix C in bovine PKA) that connects to many parts of the kinase, and its orientation is critical for activity. In the active conformation of the kinase the nearly invariant glutamate in Subdomain III forms a salt bridge with the invariant lysine of Subdomain II. This salt bridge couples subdomain III to ATP.

Subdomain IV contains a beta strand and contributes to the core structure of the small lobe.

Subdomain V contains a hydrophobic beta strand in the small lobe and an alpha helix in the large lobe. The sequence that links these two secondary structures not only links together the small and large lobes of the kinase, but also contributes residues to the ATP binding pocket and also for peptide substrate binding. In PKA Glu 127 interacts with both the ribose of ATP and the first Arg in the phosphorylation motif RRxS of a peptide substrate.

Subdomain VIa is a long alpha helix in the large lobe that parallels the alpha helix of subdomain IX.

Subdomain VIb contains the catalytic loop with the conserved motif HRDLKxxN (In PKA the H is a Y, instead). The D of this motif is the catalytic base that accepts the hydrogen removed from the hydroxyl group being phosphorylated. Note the proximity of the glutamate residue to peptide residue that will be phosphorylated, here represented by an alanine in the inhibitor peptide. A substrate peptide would contain a serine instead of the alanine, and the hydroxyl group would narrow the gap between the substrate and the glutamate.

Subdomain VII contains two beta strands link by the Mg-binding loop with the DFG motif (Aspartate Phenylalanine Glycine, or Asp Phe Gly). The Aspartate in this motif chelates a Mg²⁺ ion (Mn²⁺ in the 1ATP crystal structure) that bridges the gamma and beta phosphates of ATP and positions the gamma phosphate for transfer to the substrate.

Subdomain VIII contains several important features. The APE motif is located at the carboxyl end of this subdomain and the glutamate in this motif forms a salt bridge with an arginine in in Subdomain XI. This salt bridge is critical for forming the stable kinase core and it provides an anchor for the movement of the activation loop. In many protein kinases there is a phosphorylatable residue seven to ten residues upstream of the APE motif. In PKA it is a phosphothreonine, which forms an ionic bond with the arginine in the YRDLKPEN motif of the catalytic loop and helps to position it for catalysis. Kinases that don’t have a phosphorylatable residue in this loop often have an acidic residue that can form the salt bridge. Between the phosphorylated residue and the APE motif lies the P+1 loop, which interacts with the residue adjacent to the phosphorylated residue of the peptide substrate. The “P” residue is the one that is phosphorylated in the substrate, and the “P + 1” residue is the next residue in the sequence.

Subdomain IX is a very hydrophobic alpha helix (helix F in mammalian PKA). It contains an invariant aspartate residue that is discussed below.

Subdomain X and Subdomain XI contain three alpha helices (G, H, and I in mammalian PKA) that form the kinase core and which are involved in binding substrate proteins.

Functional structures that involve residues from more than one subdomain have been recognized by biochemical and molecular genetic studies coupled with three-dimensional structures of protein kinases.

The activation loop comprises amino acid residues between the DFG motif in subdomain VII to the APE motif in subdomain VIII. As its name implies, it is involved in switching the activity of the kinase on and off. When the phosphorylatable residue in subdomain VIII is phosphorylated, the activation loop is positioned such that the active site cleft is accessible, the magnesium loop (DFG motif) and catalytic loop (HRDLKPxxN motif) are properly positioned for catalysis, and the P+1 loop can interact with the peptide substrate. The activation loop takes on a variety of conformations in inactive kinases that disrupt one or all of these conformations.

Two hydrophobic “spines” are important for the structure of active conformation of protein kinases. They are composed of amino acid residues that are noncontiguous in the primary structure. The catalytic spine includes the adenine ring of ATP. In PKA it comprises residues A70, V57, ATP, L173, I174, L172, M128, M231, and L227, and it is directly anchored to amino end of helix F (Subdomain IX) The regulatory spine contains residues L106, L95, F185, Y164, and it is anchored to helix F via a hydrogen bond between the invariant aspartate in helix F and the backbone nitrogen of Y164. This spine is assembled in the active conformation and disorganized in inactive conformations.

The “gatekeeper” residue is a part of subdomain V (blue) and it is located deep in the ATP-binding pocket (Subdomain I with its ATP binding loop are shown in yellow). The size of the gatekeeper residue determines the size of the binding pocket, and it is thus a gatekeeper for which nucleotides, ATP analogs, and inhibitors can bind. In PKA and about 75% of all kinases it is a large residue, such as leucine, phenylalanine or methionine as seen here. In the remaining kinases, especially tyrosine kinases, the residue is larger, such as threonine or valine. The gatekeeper’s location is between the two hydrophobic spines (gatekeeper is chartreuse, catalytic spine is blue, regulatory spine is orchid). Mutation of this residue in some kinases leads to activation of the kinase via enhanced autophosphorylation of the activation loop, and the unregulated kinase activity promotes cancer. The gatekeeper’s interaction with the two spines affects the orientation of the catalytic, magnesium binding, and activation loops.

While active conformations of protein kinases are very similar, there is great variation in the inactive conformations of protein kinases, but all involve misalignment of one or more of the structures, subdomain III (C-helix in PKA) and the catalytic, magnesium binding, and activation loops.

The following is a list of human proteins containing the protein kinase domain:

AAK1; ABL1; ABL2; ACVR1; ACVR1B; ACVR1C; ACVR2A; ACVR2B; ACVRL1; ADCK1; ADCK2; ADCK3; ADCK4; ADCK5; ADRBK1; ADRBK2; AKT1; AKT2; AKT3; ALPK1; ALPK2; ALPK3; STRADB; CDK15; AMHR2; ANKK1; ARAF; ATM; ATR; AURKA; AURKB; AURKC; AXL; BCKDK; BLK; BMP2K; BMPR1A; BMPR1B; BMPR2; BMX; BRAF; BRSK1; BRSK2; BTK; BUB1; C21orf7; CALM1; CALM2; CALM3; CAMK1; CAMK1D; CAMK1G; CAMK2A; CAMK2B; CAMK2D; CAMK2G; CAMK4; CAMKK1; CAMKK2; CAMKV; CASK; CDK20; CDK1; CDK11B; CDK11A; CDK13; CDK19; CDC42BPA; CDC42BPB; CDC42BPG; CDC7; CDK10; CDK2; CDK3; CDK4; CDK5; CDK6; CDK7; CDK8; CDK9; CDK12; CDK14; CDK16; CDK17; CDK18; CDKL1; CDKL2; CDKL3; CDKL4; CDKL5; CHEK1; CHEK2; CHUK; CIT; CKB; CKM; CLK1; CLK2; CLK3; CLK4; CSF1R; CSK; CSNK1A1; CSNK1A1L; CSNK1D; CSNK1E; CSNK1G1; CSNK1G2; CSNK1G3; CSNK2A1; CSNK2A2; DAPK1; DAPK2; DAPK3; DCLK1; DCLK2; DCLK3; DDR1; DDR2; DMPK; DYRK1A; DYRK1B; DYRK2; DYRK3; DYRK4; EGFR; EIF2AK1; EIF2AK2; EIF2AK3; EIF2AK4; ELK1; EPHA1; EPHA2; EPHA3; EPHA4; EPHA5; EPHA6; EPHA7; EPHA8; EPHB1; EPHB2; EPHB3; EPHB4; ERBB2; ERBB3; ERBB4; ERN1; ERN2; FER; FES; FGFR1; FGFR2; FGFR3; FGFR4; FGR; FLT1; FLT3; FLT4; FYN; GAK; GRK1; GRK4; GRK5; GRK6; GRK7; GSK3A; GSK3B; GUCY2C; GUCY2D; GUCY2E; GUCY2F; HCK; HIPK1; HIPK2; HIPK3; HIPK4; HUNK; ICK; IGF1R; IGF2R; IKBKB; IKBKE; ILK; INSR; IRAK1; IRAK2; IRAK3; IRAK4; ITK; JAK1; JAK2; JAK3; KALRN; KDR; SIK3; KSR2; LATS1; LATS2; LIMK1; LCK; LIMK2; LRRK1; LRRK2; LYN; MAK; MAP2K1; MAP2K2; MAP2K3; MAP2K4; MAP2K5; MAP2K6; MAP2K7; MAP3K1; MAP3K10; MAP3K11; MAP3K12; MAP3K13; MAP3K14; MAP3K15; MAP3K2; MAP3K3; MAP3K4; MAP3K5; MAP3K6; MAP3K7; MAP3K8; MAP3K9; MAP4K1; MAP4K2; MAP4K3; MAP4K4; MAP4K5; MAPK1; MAPK10; MAPK12; MAPK13; MAPK14; MAPK15; MAPK3; MAPK4; MAPK6; MAPK7; MAPK8; MAPK9; MAPKAPK2; MAPKAPK3; MAPKAPK5; MARK1; MARK2; MARK3; MARK4; MAST1; MAST2; MAST3; MAST4; MASTL; MELK; MERTK; MET; MINK1; MKNK1; MKNK2; MLKL; MOS; MST1R; MST4; MTOR; MYLK; MYLK2; MYLK3; MYLK4; NEK1; NEK10; NEK11; NEK2; NEK3; NEK4; NEK5; LOC100506859; NEK6; NEK7; NEK8; NEK9; MGC42105; NLK; NRK; NTRK1; NTRK2; NTRK3; NUAK1; NUAK2; OBSCN; OXSR1; PAK1; PAK2; PAK3; PAK4; PAK6; PAK7; PASK; PBK; PDGFRA; PDGFRB; PDIK1L; PDPK1; PHKA1; PHKB; PHKG1; PHKG2; PIK3R4; PIM1; PIM2; PIM3; PINK1; PKMYT1; PKN1; PKN2; PKN3; PLK1; PLK2; PLK3; PLK4; PNCK; PRKAA1; PRKAA2; PRKACA; PRKACB; PRKACG; PRKCA; PRKCB; PRKCD; PRKCE; PRKCG; PRKCH; PRKCI; PRKCQ; PRKCZ; PRKD1; PRKD2; PRKD3; PRKG1; PRKG2; PRKX; LOC389906; PRKY; PRPF4B; PSKH1; PSKH2; PTK2; PTK2B; RAF1; RAGE; RET; RIP3; RIPK1; RIPK2; RIPK3; RIPK4; ROCK1; ROCK2; ROR1; ROR2; ROS1; RPS6KA1; RPS6KA2; RPS6KA3; RPS6KA4; RPS6KA5; RPS6KA6; RPS6KB1; RPS6KB2; RPS6KC1; RPS6KL1; RYK; SCYL1; SCYL2; SCYL3; SGK1; LOC100130827; SGK196; SGK2; SGK3; SGK494; SIK1; SIK2; SLK; SNRK; SPEG; SRC; SRPK1; SRPK2; SRPK3; STK10; STK11; STK16; STK17A; STK17B; STK19; STK24; STK25; STK3; STK31; STK32A; STK32B; STK32C; STK33; STK35; STK36; STK38; STK38L; STK39; STK4; STK40; SYK; TAOK1; TAOK2; TAOK3; TBCK; TBK1; TEC; TESK1; TESK2; TGFBR1; TGFBR2; TIE1; TIE2; TLK1; TLK2; TNIK; TNK1; TNK2; TSSK1B; TSSK2; TSSK3; TSSK4; TTBK1; TTBK2; TTK; TWF2; TXK; TYK2; TYRO3; UHMK1; ULK1; ULK2; ULK3; ULK4; VRK1; VRK2; VRK3; WEE1; WEE2; WNK1; WNK2; WNK3; WNK4; YES1; ZAK; ZAP70.

Kinases are used extensively to transmit signals and regulate complex processes in cells. Phosphorylation of molecules can enhance or inhibit their activity and modulate their ability to interact with other molecules. The addition and removal of phosphoryl groups provides the cell with a means of control because various kinases can respond to different conditions or signals. Mutations in kinases that lead to a loss-of-function or gain-of-function can cause cancer and disease in humans, including certain types of leukemia and neuroblastomas, glioblastoma, spinocerebellar ataxia (type 14), forms of a gamma-globulinaemia, and many others.

In one aspect, the therapeutic agent is a kinase inhibitor. In some aspects, the therapeutic agent is a chemotherapeutic agent. A growing interest in developing orally active protein-kinase inhibitors has recently culminated in the approval of the first of these drugs for clinical use. Protein kinases have now become the second most important group of drug targets, after G-protein-coupled receptors. Identification of the key roles of protein kinases in signaling pathways leading to development of cancer has caused pharmacological interest to concentrate extensively on targeted therapies as a more specific and effective way for blockade of cancer progression. Over the past 15 years protein kinases have become the pharmaceutical industry’s most important class of drug target in the field of cancer. Some 20 drugs that target kinases have been approved for clinical use over the past decade, and hundreds more are undergoing clinical trials.

Examples of kinase inhibitors include: Afatinib, Axitinib, Bevacizumab, Bosutinib, Cetuximab, Crizotinib, Dasatinib, Erlotinib, Fostamatinib, Gefitinib, Ibrutinib, Imatinib, Lapatinib, Lenvatinib, Masitinib, Mubritinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib and Vemurafenib. In various aspects, the chemotherapeutic agent is selected from the group consisting of dasatinib, imatinib, nilotinib, bosutinib, regorafenib, sorafenib, ponatinib, sunitinib, vermurafenib, vandetanib, ibrutinib, abemaciclib, ribociclib, palbociclib, axitinib, crizotinib, gilteritinib, erlotinib, midstaurin, ruxolitinib, brigatinib, and osimeritinib.

In various aspects, the phosphate is a DFG IN conformation, a DFG OUT conformation, or a DFG INTERMEDIATE conformation. In another aspect, the conformation of the phosphate indicates if the target is in an active state or in an inactive state. In one aspect, a DFG IN conformation of the phosphate indicates an active state of the target, a DFG OUT conformation indicates an inactive state of the target, and a DFG INTERMEDIATE conformation indicates an active state of the target.

In one aspect, a mutation in a gene encoding the target results in a change in the detection of the phosphate on the target. In some aspects, the mutation induces a lack of detection of a phosphate on DFG INTERMEDIATE conformation on the target. In other aspects, the mutation is an activating mutation or a drug resistance mutation.

In one aspect, the crystal structure library includes a kinase crystal structure database, a receptor crystal structure database, and/or a therapeutic agent crystal structure database.

In some aspects, the therapeutic agent is a drug for the treatment of cancer. In various aspects, the cancer is selected from the group consisting of an alimentary/gastrointestinal tract cancer, a liver cancer, a skin cancer, a breast cancer, an ovarian cancer, a prostate cancer, a lymphoma, a leukemia, a kidney cancer, a lung cancer, an esophageal cancer, a muscle cancer, a bone cancer, a bladder cancer, a thyroid cancer, and a brain cancer.

In another embodiment, the invention provides a method of designing a scaffold of a therapeutic agent directed against a drug-resistant target including creating a three dimensional fishing net of the distances in the DFG phosphate conformation (3D surface net) of the drug resistant target using a algorithmic phosphate detector; screening a library of small fragment to capture small fragment that specifically binds to the 3D surface net of the target, thereby identifying a scaffold structure of the therapeutic agent; and using a fragmentation algorithm to deconstruct the resistant drug structure and construct the scaffold of a therapeutic agent from the surface net structure and the captured small fragment.

In various aspects, the drug-resistant target is in a DFG INTERMEDIATE conformation, and the 3D surface net is a 3D INTERMEDIATE surface net (3D INTER net). In one aspect, creating the three-dimensional fishing net includes excluding regions of high frequency of drug resistance mutation of the kinase.

As used herein, the term “high frequency of drug resistance mutation” or “HVR region” refers to a short amino acid sequence in a kinase protein that has been discovered as the localization of multiple mutations that are responsible for drug-resistance. As discussed in the examples below, for the purposes of the therapeutic agent screening describe herein, the HVR regions have been excluded.

In some aspects, regions of high frequency of drug resistance mutation of the kinase comprises HVR regions 1-7.

In another aspect, creating the three-dimensional fishing net includes minimizing the chemical scaffold using the adenosyl ring of adenosine triphosphate (ATP).

In one aspect, screening the library includes limiting the molecular weight of the therapeutic agent to about 400 kDa.

In another aspect, the method further includes identifying analogs of the scaffold to identify the therapeutic agent.

The methods described herein allow for the identification of general scaffolds, and for the identification of therapeutic agents that fit such scaffold. As used herein, “identifying analogs” is meant to refer to the identification of known chemical compounds that fit the scaffolds described herein, and that can be, through the method described herein identified as new therapeutic agents for the described intended use. Identifying analogs can include a) applying 5 Lipinski’s rule of 5, b) considering an analog solubility; c) considering an analog commercial availability; and/or d) superposing a 3D model of an analog to a crystal structure of the scaffold.

In many aspects, the scaffold of the therapeutic agent is defined by two derivation constituents.

During the process of designing the scaffold, the methods described herein allow for the two constituents, R1 and R2 to remain free. Such components are referred to as “derivation constituents”. They are not meant to be defined to allow for more analogs to be considered, and to provide some flexibility regarding the stability of the scaffold, toxicity of the therapeutic agent, conformational specificity of the scaffold, and affinity of the therapeutic agent to the scaffold.

In one aspect, the two derivation constituents are left open to generate a combinatorial array of analogs In some aspects, a first derivation constituent (R1) improves stability of the scaffold and minimize toxicity of the therapeutic agent. In other aspects, a second derivation constituent (R2) improves conformational specificity of the scaffold and maximize affinity of the therapeutic agent to the scaffold. In one aspect, the kinase is ABL1 mutant T315I.

In one aspect, the scaffold of the therapeutic agent is selected from

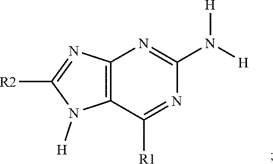

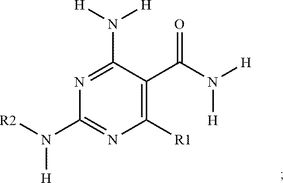

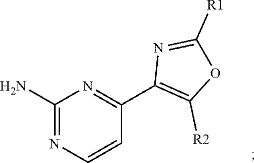

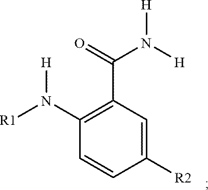

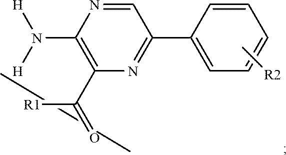

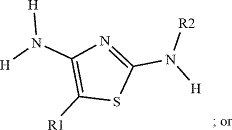

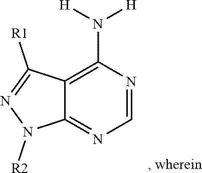

R1 and R2 are two derivation constituents.

In one embodiment, the present invention provides a method for identifying a therapeutic regimen or predicting resistance to a therapeutic regimen for a patient with cancer comprising obtaining a biologic sample from the patient; identifying at least one mutation in the gene sequence from the sample; using a pattern matching algorithm to determine if the at least one mutation is an activation mutation or a resistance mutation; and using the pattern matching algorithm and a crystal structure library to identify therapeutic agents to target the activating mutation or for which the patient is resistant; thereby identifying a therapeutic regimen or predicting resistance to a therapeutic regimen. In one aspect, the biological sample is blood, saliva, urine, bone marrow, serum, lymph, cerebrospinal fluid, sputum, stool, organ tissue or ejaculate sample. In one aspect, the at least one mutation is identified by sequence analysis. In another aspect, the at least one mutation is in the gene sequence of a receptor or a kinase. In another aspect, the at least one mutation is in the catalytic domain of a kinase. In an additional aspect, the at least one mutation results in a novel kinase conformation. In a specific aspect, the at least one mutation is in the DFG domain. In an aspect the receptor is an estrogen receptor. In certain aspects, the estrogen receptor is ESR1 or ESR2. In a further aspect, the crystal structure library comprises a protein crystal structure database and a therapeutic agent crystal structure database. In an additional aspect, the algorithm is subjected to machine learning. In one aspect, the at least one mutation comprises an activation mutation or a resistance mutation. In another aspect, the at least one mutation comprises a mutation in a kinase or a receptor. In certain aspects, the receptor is an estrogen receptor 1 (ESR1) or an estrogen receptor 2 (ESR2). In another aspect, the therapeutic regimen comprises a kinase inhibitor and/or a chemotherapeutic agent. In another aspect, the method further comprises using a three-dimensional template to identify therapeutic agents.

As used herein, the term “biological specimen” refers to any human specimen type. Examples of biological specimen include DNA, RNA, cells, tissues, organs, gametes, bodily products (teeth, hair, nail clippings, sweat, urine feces), blood and blood fractions (plasma serum red blood cells), saliva, bone marrow, lymph, cerebrospinal fluid, sputum, or ejaculate sample.

Techniques are well known in the art to detect DNA, RNA and protein mutations. Such techniques include DNA, RNA and protein sequencing.

Mutations are changes in DNA or protein sequence as compared to wild type. Mutations include insertions, deletions and point mutations. Many mutations have been identified in tumors. Identifying “actionable mutations” requires a lengthy statistical data analysis of one-dimensional genomic data gathered from many cancer patients. However, these actionable mutations are quickly outdated due to the rapid progression of the cancer. Examples of mutations identified in cancer include activating mutations and resistance mutations. Activating mutations are responsible for the onset or progression of a tumor. Resistance mutations confer resistance to the tumor to therapeutic agents rendering the therapeutic agents ineffective in treating the tumor. The mechanism of drug resistance is highly diverse and differs between patients making it difficult to determine which therapeutic agents to use in further therapy once resistance is acquired.

As used herein, the term “therapeutic regimen” refers to any course of therapy using at least one therapeutic agent in the treatment of a disease or disorder.

As used herein, the term “therapeutic agent” refers to any molecule or compound used in the treatment of a disease or disorder. The therapeutic agent maybe a kinase inhibitor. Examples of kinase inhibitor include Afatinib, Axitinib, Bevacizumab, Bosutinib, Cetuximab, Crizotinib, Dasatinib, Erlotinib, Fostamatinib, Gefitinib, Ibrutinib, Imatinib, Lapatinib, Lenvatinib, Masitinib, Mubritinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib and Vemurafenib.

Where the disease or disorder is cancer, the therapeutic agent is a chemotherapeutic drug. Examples of chemotherapeutic drugs include aromatase inhibitors, tamoxifen, Raloxifene, a competitor of estrogen in its ER binding site, antimetabolites, such as methotrexate, DNA cross-linking agents, such as cisplatin/carboplatin; alkylating agents, such as canbusil; topoisomerase I inhibitors such as dactinomycin; microtubule inhibitors such as TAXOL™ (paclitaxel), a vinca alkaloid, mitomycin-type antibiotic, bleomycin-type antibiotic, antifolate, colchicine, demecolcine, etoposide, taxane, anthracycline antibiotic, doxorubicin, daunorubicin, caminomycin, epirubicin, idarubicin, mitoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methotrexate, lomustine, thioguanine, asparaginase, vinblastine, vindesine, tamoxifen, or mechlorethamine, antibodies such as trastuzumab; bevacizumab, OSI-774, Vitaxin; alkaloids, including, microtubule inhibitors (e.g., Vincristine, Vinblastine, and Vindesine, etc.), microtubule stabilizers (e.g., Paclitaxel (TAXOL™)), and Docetaxel, Taxotere, etc.), and chromatin function inhibitors, including, topoisomerase inhibitors, such as, epipodophyllotoxins (e.g., Etoposide (VP-16), and Teniposide (VM-26), etc.), agents that target topoisomerase I (e.g., Camptothecin and Isirinotecan (CPT-11), etc.); covalent DNA-binding agents (alkylating agents), including, nitrogen mustards (e.g., Mechlorethamine, Chlorambucil, Cyclophosphamide, Ifosphamide, and Busulfan (MYLERAN™), etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); noncovalent DNA-binding agents (antitumor antibiotics), including, nucleic acid inhibitors (e.g., Dactinomycin (Actinomycin D)), anthracyclines (e.g., Daunorubicin (Daunomycin, and Cerubidine), Doxorubicin (Adriamycin), and Idarubicin (Idamycin)), anthracenediones (e.g., anthracycline analogues, such as, (Mitoxantrone)), bleomycins (Blenoxane), etc., and plicamycin (Mithramycin); antimetabolites, including, antifolates (e.g., Methotrexate, Folex, and Mexate), purine antimetabolites (e.g., 6-Mercaptopurine (6-MP, Purinethol), 6-Thioguanine (6-TG), Azathioprine, Acyclovir, Ganciclovir, Chlorodeoxyadenosine, 2-Chlorodeoxyadenosine (CdA), and 2′-Deoxycoformycin (Pentostatin), etc.), pyrimidine antagonists (e.g., fluoropyrimidines) (e.g., 5-fluorouracil (Adrucil), 5-fluorodeoxyuridine (FdUrd) (Floxuridine)) etc.), and cytosine arabinosides (e.g., Cytosar (ara-C) and Fludarabine); enzymes, including, L-asparaginase; hormones, including, glucocorticoids, such as, antiestrogens (e.g., Tamoxifen, etc.), nonsteroidal antiandrogens (e.g., Flutamide); platinum compounds (e.g., Cisplatin and Carboplatin); monoclonal antibodies conjugated with anticancer drugs, toxins, and/or radionuclides, etc.; biological response modifiers (e.g., interferons (e.g., IFN-alpha.) and interleukins (e.g., IL-2).

Cancer is a group of diseases involving abnormal cell growth with the potential to invade or spread to other parts of the body. Cancer is characterized by several biochemical mechanisms including self-sufficiency in growth signaling, insensitivity to anti-growth signals, evasion of apoptosis, enabling of a limitless replicative potential, induction and sustainment of angiogenesis and activation of metastasis and invasion of tissue.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing’s Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin’s Lymphoma, Adult; Hodgkin’s Lymphoma, Childhood; Hodgkin’s Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi’s Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin’s, Adult; Lymphoma, Hodgkin’s; Childhood; Lymphoma, Hodgkin’s During Pregnancy; Lymphoma, Non-Hodgkin’s, Adult; Lymphoma, Non-Hodgkin’s, Childhood; Lymphoma, Non-Hodgkin’s During Pregnancy; Lymphoma, Primary Central Nervous System; Macroglobulinemia, Waldenstrom’s; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin’s Lymphoma, Adult; Non-Hodgkin’s Lymphoma, Childhood; Non-Hodgkin’s Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin’s Lymphoma; Pregnancy and Non-Hodgkin’s Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing’s Family of Tumors; Sarcoma, Kaposi’s; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom’s Macro globulinemia; and Wilms’ Tumor.

A 3D pattern matching algorithm functions to analyze the 3D architecture of proteins and drug targets. Specifically, the algorithm identifies differences due to mutations or post translational modifications of a protein as well as different conformational states and unique intermediate states created by cancer activating mutations and or drug resistance mutations in a biological sample when compared to a proprietary database. For example, an algorithmic phosphate detector is a 3D pattern matching algorithm that functions to analyze 3D architecture of proteins to identify conformational changes in DFG domains, which can be translated into a phosphate DFG conformation of the protein (i.e., DFG IN conformation, DFG OUT conformation, or DFG INTERMEDIATE conformation).

The use of a proprietary crystal structure library and unique training lessons teach (i.e., machine learning) the pattern matching algorithm to predict the functionality of any kinase mutation, predict specificity of a small molecule kinase inhibitor and drug development by the prediction of virtual molecules to inhibit kinases identified by previously unknown intermediate states of kinase catalytic cores resulting from activating cancer mutations. Further, the predictive algorithm methodology enables the rapid design of new drug candidates based on the specificity profile for the predicted functionality of a mutation.

The protein crystal structure library includes the crystal structures of proteins, including kinases and receptors as well as drug ligands.

The algorithm comprises pattern matching and machine learning features to enable the accurate prediction of the functionality of the identified mutation. The analysis also enables the prediction of which therapeutic agents would target the identified mutations.

In one embodiment, the present invention relates to a method of determining risk for developing resistance or the development of resistance to a therapeutic regimen in an ER+ breast cancer patient comprising obtaining a biological sample and a tumor sample from the patient; contacting each sample with a probe that binds to a sequence in a gene associated with kinase phosphorylation; and comparing the binding of the probe in the biological sample with the binding of the probe in the tumor sample wherein binding of the probe with the biological sample but not the tumor sample is indicative of a tumor that is at risk for developing resistance to a therapeutic regimen. In one aspect, the sample is obtained from the patient following a course of therapy and wherein the course of therapy is ongoing for at least about 1 month to 6 months at the time the sample is obtained. In another aspect, the sample is obtained at intervals throughout the course of therapy. In one aspect, the subject is a human. In a further aspect, the biological sample is blood, saliva, urine, bone marrow, serum, lymph, cerebrospinal fluid, sputum, stool, organ tissue or ejaculate sample. In an additional aspect, the probe detects a mutation in the gene sequence. In a specific aspect, the mutation is a point mutation. In another aspect, the biological sample is a tumor sample and specifically, the tumor sample is a liquid biopsy or a sample of circulating tumor cells (CTCs).

In another aspect, the probe detects a deletion in the gene sequence. In one aspect, the deletion is about 2 to 12 amino acids. In a further aspect, the probe detects a deletion and a single point mutation in the gene sequence. In one aspect probe is at least about 1000 nucleotides, from about 300 to 500 nucleotides or at least about 150 nucleotides for more than one region of the gene sequence. In further aspect, the gene sequence is an ESR receptor gene sequence. In a specific aspect, the ESR receptor is ESR1 or ESR2. In certain aspects, the ESR1 receptor has a point mutation at Y537, E380, L536, and/or D538. In specific aspects the ESR1 mutation is Y537S, Y537A, Y537E or Y537K. In another aspect, the ESR2 receptor has a point mutation at V497 and specifically, the mutation is V497M.

In a further aspect, the therapeutic regimen is treatment with an aromatase inhibitor. In a specific aspect, the therapeutic regimen is treatment with a tamoxifen, Raloxifene and/or a competitor of estrogen in its ER binding site.

In another aspect, the method further comprises predicting a second form of therapy. In certain aspects, the second form of therapy is provided to the patient prior to completion of a therapeutic regimen with a first form of therapy. In another aspect, the first form of therapy is an aromatase inhibitor and the second form of therapy is a non-aromatase inhibitor chemotherapeutic drug. In an additional aspect, the non-aromatase inhibitor chemotherapeutic drug may be antimetabolites, such as methotrexate, DNA cross-linking agents, such as cisplatin/carboplatin; alkylating agents, such as canbusil; topoisomerase I inhibitors such as dactinomycin; microtubule inhibitors such as TAXOL™ (paclitaxel), a vinca alkaloid, mitomycin-type antibiotic, bleomycin-type antibiotic, antifolate, colchicine, demecolcine, etoposide, taxane, anthracycline antibiotic, doxorubicin, daunorubicin, caminomycin, epirubicin, idarubicin, mitoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methotrexate, lomustine, thioguanine, asparaginase, vinblastine, vindesine, tamoxifen, or mechlorethamine, antibodies such as trastuzumab; bevacizumab, OSI-774, Vitaxin; alkaloids, including, microtubule inhibitors (e.g., Vincristine, Vinblastine, and Vindesine, etc.), microtubule stabilizers (e.g., Paclitaxel (TAXOL™),), and Docetaxel, Taxotere, etc.), and chromatin function inhibitors, including, topoisomerase inhibitors, such as, epipodophyllotoxins (e.g., Etoposide (VP-16), and Teniposide (VM-26), etc.), agents that target topoisomerase I (e.g., Camptothecin and Isirinotecan (CPT-11), etc.); covalent DNA-binding agents (alkylating agents), including, nitrogen mustards (e.g., Mechlorethamine, Chlorambucil, Cyclophosphamide, Ifosphamide, and Busulfan (MYLERAN™), etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); noncovalent DNA-binding agents (antitumor antibiotics), including, nucleic acid inhibitors (e.g., Dactinomycin (Actinomycin D)), anthracyclines (e.g., Daunorubicin (Daunomycin, and Cerubidine), Doxorubicin (Adriamycin), and Idarubicin (Idamycin)), anthracenediones (e.g., anthracycline analogues, such as, (Mitoxantrone)), bleomycins (Blenoxane), etc., and plicamycin (Mithramycin); antimetabolites, including, antifolates (e.g., Methotrexate, Folex, and Mexate), purine antimetabolites (e.g., 6-Mercaptopurine (6-MP, Purinethol), 6-Thioguanine (6-TG), Azathioprine, Acyclovir, Ganciclovir, Chlorodeoxyadenosine, 2-Chlorodeoxyadenosine (CdA), and 2′-Deoxycoformycin (Pentostatin), etc.), pyrimidine antagonists (e.g., fluoropyrimidines (e.g., 5-fluorouracil (Adrucil), 5-fluorodeoxyuridine (FdUrd) (Floxuridine)) etc.), and cytosine arabinosides (e.g., Cytosar (ara-C) and Fludarabine); enzymes, including, L-asparaginase; hormones, including, glucocorticoids, such as, antiestrogens (e.g., Tamoxifen, etc.), nonsteroidal antiandrogens (e.g., Flutamide); platinum compounds (e.g., Cisplatin and Carboplatin); monoclonal antibodies conjugated with anticancer drugs, toxins, and/or radionuclides, etc.; biological response modifiers (e.g., interferons (e.g., IFN-alpha.) and interleukins (e.g., IL-2).

In one aspect, the determination is performed on a computer. In another aspect, the gene sequence is in a database. In a certain aspect, the database contains sequences for the catalytic cores of protein kinases.

In a further embodiment, the present invention provides a method for identifying a drug candidate comprising identifying a mutation for resistance to a first drug by genomic and/or three-dimensional crystallographic analysis; and determining a second drug based on the mutation for resistance due to the first drug, by searching a crystal structure library database to identify a scaffold for a drug candidate as the second drug, thereby identifying a drug candidate. In one aspect, a pattern matching algorithm is used to search the crystal structure library.

In another embodiment, the present invention provides a method for predicting the specificity profile of a therapeutic agent comprising obtaining the crystal structure of the therapeutic agent; and using a pattern matching algorithm to identify targets of the therapeutic agent using a crystal structure library, thereby, predicting the specificity profile of a therapeutic agent. In one aspect, the crystal structure library comprises a protein crystal structure database. In another aspect, the protein crystal structure database comprises the crystal structure of kinases and receptors. In an aspect, the therapeutic agent is a kinase inhibitor. In one aspect, the kinase inhibitor is Afatinib, Axitinib, Bevacizumab, Bosutinib, Cetuximab, Crizotinib, Dasatinib, Erlotinib, Fostamatinib, Gefitinib, Ibrutinib, Imatinib, Lapatinib, Lenvatinib, Masitinib, Mubritinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib or Vemurafenib or a combination thereof. In another aspect, the therapeutic agent is a chemotherapeutic agent. In an additional aspect, the target is a kinase or a receptor. In one aspect, the target is a mutation in a gene sequence. In a further aspect, the gene mutation is in a kinase or a receptor. In certain aspects, the target is the catalytic domain of a kinase. In a specific aspect, the target is the DFG domain. In one aspect, the receptor is an estrogen receptor. In an additional aspect, the specificity profile is used in the selection of a treatment regimen for a patient in need thereof.

In a further embodiment, the present invention provides a method of treating a patient in need thereof comprising obtaining a biologic sample; identifying at least one mutation in a gene from the biologic sample; using a pattern matching algorithm and a crystal structure library to identify at least one therapeutic agent to target the at least one mutation; and administering the identified therapeutic agent to the patient, thereby treating the patient. In one aspect, the patient is diagnosed with cancer. In another aspect, at least 2 gene mutations are identified. In certain aspects, 2, 3, 4, 5, 6, 7, 8, 9, or 10 gene mutations are identified. In a further aspect, the gene mutations are identified by sequence analysis. In an aspect, the crystal structure library comprises the crystal structure of kinases, receptors and ligands. In one aspect, the target is a kinase or a receptor. In an additional aspect, more than one therapeutic agent is selected for the treatment regimen. In a further aspect the at least one chemotherapeutic agent. In certain aspects, one chemotherapeutic agent is a kinase inhibitor. In another aspect, the method further comprises using a three-dimensional template to identify at least one therapeutic agent.

In a further embodiment, the invention provides for a method of determining a disease state in a subject comprising obtaining a biological sample and a sample suspected of containing diseased cells from the subject; contacting each sample with a probe that binds to a sequence in a gene associated with kinase phosphorylation; and comparing the binding of the probe in the biological sample with the binding of the probe in the diseased cell sample wherein binding of the probe with the biological sample but not the diseased cell sample is indicative of a disease state or risk for developing a disease state in a subject. In one aspect, the disease state may be cancer, autoimmunity, infectious disease, and genetic disease. In an aspect, the method further comprises identifying a disease therapy, monitoring treatment of a disease state, determining a therapeutic response, identifying molecular targets for pharmacological intervention, and making determinations such as prognosis, disease progression, response to particular drugs and to stratify patient risk. In an additional aspect, the method further comprises determining a proliferation index, metastatic spread, genotype, phenotype, disease diagnosis, drug susceptibility, drug resistance, subject status and treatment regimen. In another aspect, the biological sample is blood, saliva, urine, bone marrow, serum, lymph, cerebrospinal fluid, sputum, stool, organ tissue, ejaculate sample, an organ sample, a tissue sample, an alimentary/ gastrointestinal tract tissue sample, a liver sample, a skin sample, a lymph node sample, a kidney sample, a lung sample, a muscle sample, a bone sample, or a brain sample, a stomach sample, a small intestine sample, a colon sample, a rectal sample, or a combination thereof. In a further, aspect, the cancer is selected from an alimentary/gastrointestinal tract cancer, a liver cancer, a skin cancer, a breast cancer, an ovarian cancer, a prostate cancer, a lymphoma, a leukemia, a kidney cancer, a lung cancer, an esophageal cancer, a muscle cancer, a bone cancer, or a brain cancer. In certain aspects, the cancer is breast cancer and the breast cancer is ER+ breast cancer. In an aspect, the drug is a chemotherapeutic drug, an antibiotic, or an anti-inflammatory drug. In another aspect, the subject is a mammal and specifically, the human subject is a human.

In an additional embodiment, the present invention provides for a system for automated determination of an effective protein kinase inhibitor drug for a patient in need thereof comprising an input operable to receive patient sequence data for a protein kinase suspected of being associated with a disease state; a processor configured to apply the received sequence data to a first database comprising three-dimensional models of crystal structures of protein kinases, the processor configured to provide a display aligning a native protein kinase with the patient’s protein kinase sequence, thereby identifying a region in the three-dimensional crystal structure of the kinase where the patient’s kinase differs from the native kinase. In one aspect, the method further comprises a processor for input from a second database, wherein the second database comprises a plurality of protein kinase inhibitor drugs, thereby allowing stratification of one or more drug treatment options in a report based on the output status of the patient sequence data and the protein kinase inhibitor drugs. In an additional aspect, the patient is a cancer patient. In another aspect, the kinase is a tyrosine kinase.

In one embodiment, the present invention provides for a method of determining a therapeutic regimen for a patient comprising utilizing the system described above to determine one or more drugs for which the patient will be responsive and administering the one or more drugs to the patient based on the stratifying. In another aspect, the stratifying further comprises ranking one or more drug treatment options with a higher likelihood of efficacy or with a lower likelihood of efficacy. In another aspect, the stratifying further comprises ranking one or more drug treatment options with a higher likelihood of developing drug resistance of a lower likelihood of developing drug resistance. In a further aspect, the stratifying is indicated by color coding the listed drug treatment options on the report based on a rank of a predicted efficacy or resistance of the drug treatment options. In one aspect, the annotating comprises using information from a commercial database. In a further aspect, the annotating comprises providing a link to information on a clinical trial for a drug treatment option in the report. In one aspect, the annotating comprises adding information to the report selected from the group consisting of one or more drug treatment options, scientific information regarding one or more drug treatment options, one or more links to scientific information regarding one or more drug treatment options, one or more links to citations for scientific information regarding one or more drug treatment options, and clinical trial information regarding one or more drug treatment options.

In an additional embodiment, the present invention provides for a system for automated determination of an effective protein kinase inhibitor drug for a patient in need thereof comprising a database; and a processor circuit in communication with the database, the processor circuit configured to receive patient sequence data for a protein kinase suspected of being associated with a disease state; identify data indicative of a disease state within the database; store the data indicative of the disease state in the database; organize the data indicative of the disease state based on disease state; analyze the data indicative of the disease state to generate a treatment option based on the disease state and protein kinase inhibitor drug; and cause the treatment option and the organized data to be displayed.

In a further embodiment, the present invention provides for a method of determining a second course of therapy for a subject having developed resistance for a first course of therapy comprising identifying a mutation for resistance to the first course of therapy by genomic and/or three-dimensional crystallographic analysis; and determining a drug for the second course of therapy based on a search of a database of existing drugs, thereby identifying the second course of therapy. In one aspect, the method further comprises preparing nucleic acid-based probes that correlate with the mutation for the resistance to the first course of therapy.

In one embodiment, the present invention provides for a method of determining a second course of therapy for a subject having developed resistance for a first course of therapy comprising identifying a mutation for resistance to the first course of therapy by genomic and/or three-dimensional crystallographic analysis; and determining a drug for the second course of therapy based on a search of a crystal structure library database to identify a scaffold for a drug candidate as the second course of therapy, thereby identifying the second course of therapy. In an aspect, the determining step uses a quantum computer.

In another embodiment, the present invention provides for a method for identifying a drug candidate comprising identifying a mutation for resistance to a first drug by genomic and/or three-dimensional crystallographic analysis; and determining a second drug based on the mutation for resistance due to the first drug, by searching a crystal structure library database to identify a scaffold for a drug candidate as the second drug, thereby identifying a drug candidate.

In one embodiment, the invention provides a method of treating cancer in a subject including administering to the subject a therapeutically effective amount of a therapeutic agent, wherein the cancer is characterized by a mutation in an ABL1 gene, and wherein the therapeutic agent is selected from

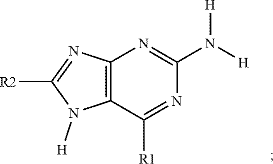

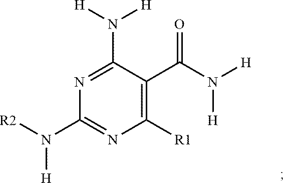

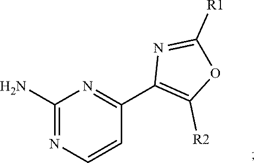

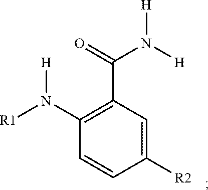

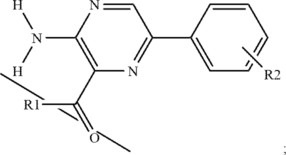

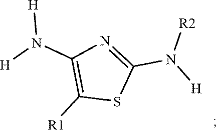

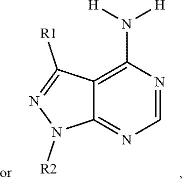

, wherein R1 and R2 are two derivation constituents, thereby treating cancer in the subject.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/ preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of a leukemia or treatment of a drug-resistant leukemia). Such amount should be sufficient to treat leukemia in the subject. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal , oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In some aspects, the cancer is characterized by a mutation in an ABL1 gene.

Tyrosine-protein kinase ABL1 also known as ABL1 is a protein that, in humans, is encoded by the ABL1 gene located on chromosome 9. c-Abl is sometimes used to refer to the version of the gene found within the mammalian genome, while v-Abl refers to the viral gene, which was initially isolated from the Abelson murine leukemia virus. Mutations in the ABL1 gene are associated with chronic myelogenous leukemia (CML). In CML, the gene is activated by being translocated within the BCR (breakpoint cluster region) gene on chromosome 22. This new fusion gene, BCR-ABL, encodes an unregulated, cytoplasm-targeted tyrosine kinase that allows the cells to proliferate without being regulated by cytokines. This, in turn, allows the cell to become cancerous. This gene is a partner in a fusion gene with the BCR gene in the Philadelphia chromosome, a characteristic abnormality in chronic myelogenous leukemia (CML) and rarely in some other leukemia forms. The BCR-ABL transcript encodes a tyrosine kinase, which activates mediators of the cell cycle regulation system, leading to a clonal myeloproliferative disorder. The BCR-ABL protein can be inhibited by various small molecules.

Cancer cells are known to develop various mechanism to escape the efficacy of anti-cancer drug, including TKIs. Mutations in the kinase domain (KD) of BCR-ABL are the most prevalent mechanism of acquired resistance to TKIs in patients with chronic myeloid leukemia (CML). Specifically, some punctual mutation in the sequence of BCR-ABL have been shown to be associated with a resistance to TKIs such as imatinib.

In some aspects, the ABL1 mutation is T315I.

In one aspect, the cancer is resistant to one or more tyrosine kinase inhibitors (TKIs). In some aspects, the one or more TKIs are selected from the group consisting of imatinib, nilotinib, dasatinib, bosutinib, ibrutinib and ponatinib. In other aspects, the ABL1 mutation is T315I.

In another embodiment, the present invention provides a method of treating leukemia in a subject including administering to the subject a therapeutically effective amount of a cyclin dependent kinase (CDK) inhibitor, wherein the CDK inhibitor is NU6027, thereby treating leukemia in the subject.

Leukemia is a group of cancers that develops in the early blood-forming cells, that usually begins in the bone marrow and that results in high numbers of abnormal blood cells, which are not fully developed and called blasts or leukemia cells. Most often, leukemia is a cancer of the white blood cells, but some leukemias start in other blood cell types. There are several types of leukemia, which are divided based mainly on whether the leukemia is acute (fast growing) or chronic (slower growing), and whether it starts in myeloid cells or lymphoid cells. There are four main types of leukemia—acute lymphoblastic leukemia (ALL), acute myeloid leukemia (AML), chronic lymphocytic leukemia (CLL) and chronic myeloid leukemia (CML)—as well as a number of less common types. Leukemias and lymphomas both belong to a broader group of tumors that affect the blood, bone marrow, and lymphoid system, known as tumors of the hematopoietic and lymphoid tissues. The exact cause of leukemia is usually unknown, with the combination of genetic factors and environmental (non-inherited) factors believed to play a role. Risk factors include smoking, ionizing radiation, some chemicals (such as benzene), prior chemotherapy, and Down syndrome. People with a family history of leukemia are also at higher risk. Different types of leukemia have different treatment options and outlooks. Treatment may involve some combination of chemotherapy, radiation therapy, targeted therapy, and bone marrow transplant, in addition to supportive care and palliative care as needed. The success of treatment depends on the type of leukemia and the age of the person. CML was the first cancer to be linked to a clear genetic abnormality, the chromosomal translocation known as the Philadelphia chromosome, responsible for the fusion of part of the BCR (“breakpoint cluster region”) gene from chromosome 22 with the ABL gene on chromosome 9. This abnormal fusion gene generates a Bcr-Abl fusion protein carrying a tyrosine kinase domain capable of activating a cascade of proteins that control the cell cycle and speed up cell division. Targeted therapies that specifically inhibit the activity of the Bcr-Abl protein can induce complete remissions in CML, confirming the central importance of Bcr-Abl as the cause of CML. Bcr-Abl specific inhibitors are tyrosine kinase inhibitors (TKIs) a pharmaceutical drug that inhibits tyrosine kinases. Tyrosine kinases are enzymes responsible for the activation of many proteins by signal transduction cascades. The proteins are activated by adding a phosphate group to the protein (phosphorylation), a step that TKIs inhibit. While the outcomes of leukemia have improved in the developed world, with a five-year survival rate is 57% in the United States (in children under 15, the five-year survival rate is greater than 60% or even 90%, depending on the type of leukemia), and while in children with acute leukemia who are cancer-free after five years, the cancer is unlikely to return, many patients develop drug resistance. This is often the result of the acquisition of cancer cell mutations that render a targeted drug unable to bind to its target, and results in recurrence and/or progression of the disease, which is usually associated with unfavorable outcomes.

As used herein, the term “leukemia” refers to a group of cancer that develops in the early blood-forming cells. Clinically and pathologically, leukemia can be subdivided into a variety of large groups. The first division being between its acute and chronic forms. Acute leukemia is characterized by a rapid increase in the number of immature blood cells. The crowding that results from such cells makes the bone marrow unable to produce healthy blood cells resulting in low hemoglobin and low platelets. Immediate treatment is required in acute leukemia because of the rapid progression and accumulation of the malignant cells, which then spill over into the bloodstream and spread to other organs of the body. Acute forms of leukemia are the most common forms of leukemia in children. Chronic leukemia is characterized by the excessive buildup of relatively mature, but still abnormal, white blood cells. Typically taking months or years to progress, the cells are produced at a much higher rate than normal, resulting in many abnormal white blood cells. Whereas acute leukemia must be treated immediately, chronic forms are sometimes monitored for some time before treatment to ensure maximum effectiveness of therapy. Chronic leukemia mostly occurs in older people but can occur in any age group. Additionally, the diseases are subdivided according to which kind of blood cell is affected. This divides leukemias into lymphoblastic or lymphocytic leukemias and myeloid or myelogenous leukemias. In lymphoblastic or lymphocytic leukemias, the cancerous change takes place in a type of marrow cell that normally goes on to form lymphocytes, which are infection-fighting immune system cells. Most lymphocytic leukemias involve a specific subtype of lymphocyte, the B cell. In myeloid or myelogenous leukemias, the cancerous change takes place in a type of marrow cell that normally goes on to form red blood cells, some other types of white cells, and platelets. Combining these two classifications provides a total of four main categories. Within each of these main categories, there are typically several subcategories. Finally, some rarer types are usually considered to be outside of this classification scheme.

In some aspects, the leukemia is selected from the group consisting of acute lymphoblastic leukemia (ALL), acute myeloid leukemia (AML), blastic plasmacytoid dendritic cell neoplasm (BPDCN), chronic lymphocytic leukemia (CLL), chronic myelogenous leukemia (CML), hairy cell leukemia, mast cell leukemia and meningeal leukemia.

Acute lymphoblastic leukemia (ALL) is the most common type of leukemia in young children. It also affects adults, especially those 65 and older. Standard treatments involve chemotherapy and radiotherapy. Subtypes include precursor B acute lymphoblastic leukemia, precursor T acute lymphoblastic leukemia, Burkitt’s leukemia, and acute biphenotypic leukemia. While most cases of ALL occur in children, 80% of deaths from ALL occur in adults.

Chronic lymphocytic leukemia (CLL) most often affects adults over the age of 55. It sometimes occurs in younger adults, but it almost never affects children. Two-thirds of affected people are men. The five-year survival rate is 85%. It is incurable, but there are many effective treatments. One subtype is B-cell prolymphocytic leukemia, a more aggressive disease.

Acute myelogenous leukemia (AML) occurs far more commonly in adults than in children, and more commonly in men than women. It is treated with chemotherapy. The five-year survival rate is 20%. Subtypes of AML include acute promyelocytic leukemia, acute myeloblastic leukemia, and acute megakaryoblastic leukemia.

Chronic myelogenous leukemia (CML) occurs mainly in adults; a very small number of children also develop this disease. It is treated with tyrosine kinase inhibitor such as imatinib or other drugs. The five-year survival rate is 90%. One subtype is chronic myelomonocytic leukemia.

Hairy cell leukemia (HCL) is sometimes considered a subset of chronic lymphocytic leukemia but does not fit neatly into this category. About 80% of affected people are adult men. No cases in children have been reported. HCL is incurable but easily treatable. Survival is 96% to 100% at ten years.

T-cell prolymphocytic leukemia (T-PLL) is a very rare and aggressive leukemia affecting adults; somewhat more men than women are diagnosed with this disease. Despite its overall rarity, it is the most common type of mature T cell leukemia; nearly all other leukemias involve B cells. It is difficult to treat, and the median survival is measured in months.

Large granular lymphocytic leukemia may involve either T-cells or NK cells; like hairy cell leukemia, which involves solely B cells, it is a rare and indolent (not aggressive) leukemia.

Adult T-cell leukemia is caused by human T-lymphotropic virus (HTLV), a virus similar to HIV. Like HIV, HTLV infects CD4+ T-cells and replicates within them; however, unlike HIV, it does not destroy them. Instead, HTLV “immortalizes” the infected T-cells, giving them the ability to proliferate abnormally. Human T-cell lymphotropic virus types I and II (HTLV-I/II) are endemic in certain areas of the world.

In some aspects, the leukemia is CML.

While the illustrative example in the present disclosure relates to treatment of leukemia, it should be understood that A-0001 can be used to treat other cancers related to ABL1 mutations. As further detailed below, ABL1 mutations are associated with uncontrolled proliferation in cells, which is a hallmark of cancer. ABL1 mutations have been described in a variety of cancer types, therefore invention drugs can inhibit mutated ABL1 kinase for the treatment of any such cancer. Accordingly, A-0001 can be used to treat subjects with cancers including hematological cancers (including leukemia, lymphomas, myelodysplastic syndromes and myeloproliferative neoplasms), as well as for lung, colon, endometrial, breast, prostate, bladder, ovarian, rectal, pancreatic, esophageal cancers, melanoma and glioblastoma, for example.

As used herein, “NU6027” can be referred to as “A-0001”, or “CAS 220036-08-8” without any difference in the meaning, and refers to the compound 6-Cyclohexylmethyloxy-5-nitroso-pyrimidine-2,4-diamine having the molecular formula C₁₁H₁₇N₅O₂, and the chemical formula

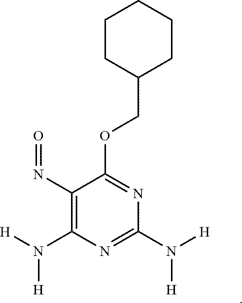

NU6027 is a selective cyclin-dependent kinase 2 (CDK2) inhibitor and a potent inhibitor of ATR signaling. NU6027 inhibits the growth of human tumor cells with mean GI50 of 10 µM. NU6027 causes a reduction cancer cell survival and proliferation by reducing the number of cells in S-phase (without affecting the number of cells in G1 or G2/M). NU6027 is a potent inhibitor of cellular ATR activity with IC50 of 6.7 µM in MCF7 cells and 2.8 µM in GM847KD cells and enhances hydroxyurea and cisplatin cytotoxicity in an ATR-dependent manner.

Cyclin-dependent kinase 2, also known as cell division protein kinase 2, or Cdk2, is an enzyme that in humans is encoded by the CDK2 gene, and a member of the cyclin-dependent kinase family of Ser/Thr protein kinases. Cdk2 is a catalytic subunit of the cyclin-dependent kinase complex, whose activity is restricted to the G1-S phase of the cell cycle, where cells make proteins necessary for mitosis and replicate their DNA. This protein associates with and is regulated by the regulatory subunits of the complex including cyclin E or A. Cyclin E binds G1 phase Cdk2, which is required for the transition from G1 to S phase while binding with Cyclin A is required to progress through the S phase. Its activity is also regulated by phosphorylation.

NU6027 can be administered alone or in combination with a pharmaceutically acceptable carrier. By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington’s Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

Alternatively, a pharmaceutically acceptable salt of NU6027 can be administered. The term “pharmaceutically acceptable salts” refers to physiologically and pharmaceutically acceptable salts of the compounds of the invention, e.g., salts that retain the desired biological activity of the parent compound and do not impart undesired toxicological effects thereto.

As described herein, NU6027 is used for the treatment of leukemia and is therefore used for its protein tyrosine kinase inhibitory activity. Protein tyrosine kinase (PTK) is one of the major signaling enzymes in the process of cell signal transduction; it catalyzes the transfer of ATP-γ-phosphate to the tyrosine residues of the substrate protein (i.e., phosphorylating the protein), a process involved in the regulation of cell growth, differentiation, death and a series of physiological and biochemical processes. Abnormal expression of PTK usually leads to cell proliferation disorders, and is closely related to tumor invasion, metastasis and tumor angiogenesis. A variety of PTKs are used as targets in the screening of anti-tumor drugs. Tyrosine kinase inhibitors (TKIs) compete with ATP for the ATP binding site of PTK and reduce tyrosine kinase phosphorylation, thereby inhibiting cancer cell proliferation. The anti-tumor mechanism of TKI can be achieved by inhibiting the repair of tumor cells, blocking the cell division in G1 phase, inducing and maintaining apoptosis, anti-angiogenesis and so on.

TKIs are responsible for great progress in the treatment of cancer, but acquired resistance is inevitable, restricting the efficacy of the treatment of cancer. Even in highly sensitive patients with TKI, tumor cells can always self-adjust, look for a way out, to avoid TKI target, which ultimately can lead to acquired resistance and disease progression. As a result, most TKI therapies are only effective for a limited period of time.

Since their discovery, many TKIs having various PTK targets have been developed and approved for the treatment of cancer (Table 1).

TABLE 1

Non-exhaustive list of approved TKIs and their target(s)

| | |
|---|---|
| TKI | Target |
| Imatinib | Abl, PDGFR, SCFR |
| Gefitinib | EGFR |
| Nilotinib | Bcr-Abl, PDGFR |
| Sorafenib | Raf, VEGFR, PDGER |
| Sunitinib | PDGFR, VEGFR, |
| Dasatinib | Bcr-Abl, SRC, PDGFR |
| Lapatinib | EGFR |
| Pazopanib | VEGFR, PDGFR, FGFR |
| Crizotinib | ALK |
| Ruxolitinib | JAK1, JAK2 |
| vandetanib | VEGFR, EGFR |
| Axitinib | VEGFR |
| Bosutinib | Abl, SRC |
| Afatinib | EGFR |
| Erlotinib | EGFR |
| Ceritinib | ALK |
| Osimertinib | EGFR |
| Lenvatinib | VEGFR |
| Alectinib | ALK |
| Regorafenib | VEGFR, EGFR |
| Neratinib | HER2 |
| Brigatinib | ALK |
| Ibrutinib | BTK |
| Ponatinib | BCR-ABL |

In one aspect, the leukemia is resistant to one or more tyrosine kinase inhibitors (TKIs). In some aspects, the one or more TKIs are selected from the group consisting of imatinib, nilotinib, dasatinib, bosutinib, ibrutinib and ponatinib.

In most cases, there is no identified cause for leukemia. However, in some cases leukemia can be characterized by a genetic mutation leading to the uncontrolled proliferation of blood cells.

Drug-resistance can be innate (cancer cells are inherently resistant to the drug) or acquired (cancer cells become resistant to the drug after exposition to the drug). Acquired drug-resistance can be the result of the acquisition by the cancer cells of a drug-resistant mutation, which usually arises after exposure of a cancer cell to the drug and is a cancer mechanism to escape the mechanism of action of the drug. In various aspects, an acquired drug-resistance arise while or after the subject having cancer is treated with the drug.

In one aspect, the subject has previously been treated with imatinib, nilotinib, dasatinib, ibrutinib, ponatinub, or a combination thereof.

Drug-resistant mutations, such as ABL1 T315I mutation can induce change to the phosphorylation state of a protein, which in turn can impact the ability of a TKI to bind to a tyrosine kinase domain.

In one aspect, NU6027 binds to a mutated ABL1 kinase domain in either a phosphorylated or unphosphorylated conformation.

The efficacy of a drug, such as a TKI may be evaluated by a measure of the specific binding of the TKI to its target. As used herein, “specific binding” refers to TKI binding to a target tyrosine kinase domain (TKD). Typically, a TKI specific binding can be measured by a K_{D} of the TKI. The term “kd” (sec⁻¹), as used herein, is intended to refer to the dissociation rate constant of a particular TKI/TKD interaction. This value is also referred to as the off value. The term “K_{D”} (M-1), as used herein, is intended to refer to the dissociation equilibrium constant of a particular TKI/TKD interaction.

Mutated TKDs that are resistant to TKI often present a reduced K_{D} as compared to the K_{D} or a non-mutated TKD, which explains the loss of efficacy of the TKI. NU6027, as described herein has a greater binding to mutated TKD than known TKIs.

In some aspects, NU6027 binds to a mutated ABL1 kinase domain with a K_{D} that is at least 100 times greater that the K_{D} of imatinib, nilotinib, dasatinib, bosutinib, ibrutinib or ponatinib.

For example, K_{D} of NU6027 for a mutated ABL1 kinase can be 10x, 20x, 30x, 40x, 50x, 60x, 70x, 80x, 90x, 100x, 150x, 200x, 250x or more times greater than the K_{D} of imatinib, nilotinib, dasatinib, bosutinib, ibrutinib or ponatinib for a mutated ABL1 kinase.

In another embodiment, the invention provides a method of treating a drug-resistant chronic myelogenous leukemia (CML) in a subject comprising administering to the subject a therapeutically effective amount of NU6027, thereby treating the drug-resistant CML in the subject.

In one aspect, the CML is resistant to imatinib, nilotinib, dasatinib, bosutinib and/or ponatinib.

The invention in all its aspects is illustrated further in the following Examples. The Examples do not, however, limit the scope of the invention, which is defined by the appended claims.

### EXAMPLES

### Example I

### Construction of a Human Protein Kinase Library

A library was constructed of all the human protein kinase structures that have been published in the Protein Data Bank. The database provided information regarding any mutations in the kinase, the location of any mutations within the three-dimensional structure of the kinase as well as whether an approved drug has been crystalized with a kinase and an associated mutation. The library was assembled using a DNA SEQ script. The DNA SEQ script can be run (used) on the Protein Data Bank (PDB). All available PDB files that contain a human kinase structure are “pruned” (term “pruning” is referred to alteration of PDB file in very unique way) and aligned to the first crystal structure of protein kinase that is 1ATP. The script divides the protein from the ligand. The final library has the following structural files:

ZZxxxxx that represents all the protein kinases aligned (using DNA SEQ script).

AAxxxxx that represent all the ligands generated from co-crystallization to human kinase all aligned as the complex (ligand and kinase).

YYxxxxx is the alignment of all APO (no ligands) structures find among human kinase crystallized.

The key optimization problem set for the algorithm is to “reconstruct the complex from ZZxxxxx file and AAxxxxx file. During the process of reconstruction’ the various criteria are being used which, in general, can be defined as “the teaching lessons”. Correct reconstruction of the complex through a set of lessons provides the algorithm the path to learn (see [0178]).

This database provides guidance as to whether a mutation will interfere with the binding of a drug or clinical candidate for a kinase and predict a known drug or clinical candidate that should be used for that mutation. The database includes a functional alignment to a kinase structure that contains information regarding conformation close to the active state (i.e., active kinase conformation, ATP, ions, substrate and regulatory domain) to provide structure/function perspective. This database has been utilized to provide therapeutic recommendations, identify a potential risk factor, develop predictive guidance on previously known mutations and kinases for which the structure is unknown and drug development.

In one example, 2,139 crystal structures of human protein kinase catalytic domains were extracted from the database and aligned to the 1ATP crystal structure. Diverse kinase structures were overlayed and the resulting alignment at the ATP binding pocket was analyzed. Three key regions were analyzed: the hinge region, DFG specificity pocket and the ATP substrate.

Once a kinase mutation was identified by sequencing, the database was queried to determine if the structure of the kinase is known; if a structure with that mutation is known; if a structure of the kinase that contains bound ligands is known and if there is a clinical drug structure known, for either the wild type or mutated kinase. From this information guidance was derived for determining recommendations for mutation responsive/nonresponsive drug treatments.

In another example, the library was refined by analyzing the 2,139 aligned protein kinases for their rmsd versus the 1 ATP reference. RMSD is a specific parameter routinely used by crystallographers that represents: Root-Mean-Square Deviation of atomic positions. Deviation from two structures (specifically two atoms with each distinct XYZ positions) being compared - please refer to: en.wikipedia.org/wiki/Root-mean-square deviation of atomic positions.

In this example we compare the staurosporine, a nonspecific ligand for kinases (binds all), versus imatinib (Gleevec®) the specific ligand that binds to the specific conformation of the kinase targets (c-abl.c-kit and PDGF).

The 718,704 rmsd values were then averaged for each of the 336 residues in 1ATP. The average rmsd values were plotted against the sequence numbering. The kinase library was analyzed for overall similarity. Sequence rmsd cutoffs were used to truncate the alignment, the model altered the alignment of the ligand staurosporine. Structural impact of ligand binding, kinase library similarity-complexes, kinase library similarity - unliganded, staurosporine complexes only and imatinib (STI) complexes only were analyzed.

### Example II

### A 3D Pattern Matching Machine Learning Algorithm

The 3D pattern matching machine learning algorithm was developed using similar structures to define interactions, maximum common subgraph problem, reduction to a maximum clique problem, and branch and bound based algorithms.

The first objective was to compute MCS for every pair of molecules in the dataset; finds groups of “similar” molecules; represent the data set visually in a 3D space, so that “similar” molecules would be close to one another. MCS=Maximum Common Sub Graph. This definition is currently being used in pattern matching and machine learning. In a simple way it means that if man or women is perfectly dressed- the key elements combining and creating maximum Common Sub graph of Elegance/Style, must include shoes, bag, dress, watch. Nobody cares about his/her underwear. Some designers/mathematician will eliminate the watch. Then if we run millions of them defining the maximum common sub graph we might end up with shoes only, bags only, or dress only, but if we define the criteria better (this our teaching lesson) we end up with the top dressed man/women in the world.

Grouping was done by using a spectral clustering algorithm; embedding was done by solving the following problem:

$$\begin{matrix}\text{Min} \sum \left(\left‖\text{xi-xj}\right‖ \text{2-dij}\right) 2 \\ \text{x} \text{ij}\end{matrix}$$

The second objective was to modify the subgraph criteria. This refers to the small molecule: a subgraph is just a way to simplify a molecule in an object that is simple to run with an algorithm, and to easily recall the molecule, or class of molecules, is derived from. (Watch, shoes beg, dress or watch only), making it less restrictive; instead of looking at all the pairs of atoms look only at the close neighbors. Maximum clique-based algorithm does not work at this point.

The third objective was to split the data set into two groups based on similarity to a given two molecules and then to split each group further into subgroups based on molecules mutual similarities in each group.

The fourth objective was to find patterns in molecules localized in space and specific locations (such as presence of N-C-N pattern). When looking for similarities between molecules this localization was imposed as an additional constraint. Subgraphs were developed based on distance threshold, maximum connected components and nearest atom idea,

The algorithm was then optimized by finding a pattern that optimizes a certain function:

$$\begin{matrix}min E \left(\text{s}\right) + \text{R} \left(\text{s}\right) \\ \text{s,} \left|\text{s}\right| \text{=k}\end{matrix}$$

Thereafter, a two stage Tabu search was performed to find a pattern minimizing E(s); within proximity of found pattern find a pattern that minimizes R(s); weight differently atoms of different element types; expand resulting subgraphs few steps along the connections. Step 1 consisted of picking few nearest atoms and connect with shortest paths. Step 2 consisted of running a stage 1 Tabu search. The final step consisted of running a stage 2 Tabu search.

The algorithm was further optimized by Machine learning. The problem of Machine learning can generally be formulated as follows: given a set of objects X, a set of labels Y and an objective function:

$$\text{y} \text{*} \text{:} \text{X} \to \text{Y;}$$

that maps objects from X to labels from Y; values yi=y*(xi) are known for the limited subset of objects

$$\left\{{\text{X}}_{1} … … , {\text{X}}_{1}\right\} ⊂ \text{X;}$$

called training set; the task is based on the training set to construct an algorithm a: X→Y satisfying: an algorithm should allow efficient computational implementation and the algorithm should be able to correctly reconstruct labels on the training set: a(xi)=yi, i=1, ......,l. The equality can be approximate, and the algorithm should have a generalization ability, meaning it should be able to identify with a high accuracy labels on the elements from X that do not belong to the training set (the elements that algorithm has not “seen” before).

This machine learning algorithm was applied to a molecular interaction problem. Here each object is a pair of molecules and each label is a binary value, indicating whether given two molecules interact with each other. The set X is therefore a set of all pairs of molecules, and the set Y contains information on whether molecules from each particular pair interact with each other or not. The training set is essentially data for which the answers are known: a set of pairs of molecules, for which it is known, whether they interact or not. The objective is to design an algorithm, which by learning from the training set, was able to apply obtained knowledge to identify with a high accuracy whether any arbitrary pair of molecules would interact. The following items were crucial in order to successfully solve the machine learning problem: 1) a good training set. A clean high-quality set of molecules, for which we are confident in the correct answers. Usually, the larger the training set is, the better is the resulting algorithm, since there is more information for it to learn from. 2) a good representation of objects, which in this case are molecules. The naive straightforward representations (such as encoding each molecule as a sequence of its atoms with coordinates for each atom) usually don’t work. A set of insightful features must be identified from which the algorithm would be able to efficiently learn. 3) finally, in order to test and compare the algorithm, a small testing set of molecules for which there is known answers, but which will not be part of the training set is needed. The algorithm was run on this test data set and then the predictions were compared to the known answers. The algorithm has the possibility to enter the process of machine learning if it is provided with a statistical series of data to train the algorithm in advance. If yes, the algorithm uses a machine learning process, if not algorithm can give an immediate answer based on instructions.

The algorithm was used to classify receptors based on a kinase DFG domain pattern. Three different DFG patterns classes were identified: in, out and intermediate (inter).

Handling dual conformations. The DFG motif as discovered in PDB: 1ATP exists in two major conformations IN and OUT. The IN conformation IN kinase is active and sends signals to the network, in the OUT conformation the kinase is inactive and does not send the signal to the network. Identification of INTER using our algorithm and machine learning process is the single most significant accomplishment of this methodology leading to a novel way of designing a small molecules oncology drug.

DFG classification geometrical features for machine learning were identified. Generalized additive models were used:

$$log \frac{μ \left(\text{X}\right)}{1 - μ \left(\text{X}\right)} = α + {\text{s}}_{1} \left({\text{X}}_{\text{1}}\right) + … + {\text{s}}_{\text{m}} {\left({\text{X}}_{\text{m}}\right)}_{1}$$

µ(X)=P(Y=1|X)
Y∈{0,11 is a class;
X=(X.,...,X..) are the features
S is a nonlinear functions associated with the i-th feature
α is a free term

Estimations of risk functions for continuous features function si(x) are estimated by fitting natural cubic splines (piecewise polynomial functions):

$$\text{minRSS} \left(\text{f} , λ\right) = {{\sum }_{4 - 1}^{\text{n}} \left({\text{y}}_{\text{1}} \text{-f} \left({\text{x}}_{\text{1}}\right)\right)}^{2} + λ {\sqrt{\text{f}}}^{′ \text{} ′} \left(\text{t}\right) \text{dt}$$

the degrees of freedom (complexity) of the splines are learned in the training process by maximizing the restricted likelihood function; all computations ere done using R mgcv package. Fitting additive models was performed using the following:

The Backfitting Algorithm for Additive Models
1. Initialize: $\hat{α} = \frac{1}{N} \sum {}_{1}^{N} {y}_{i} , {f}_{3} ≡ 0 , {∀}_{i} , j .$
2. Cycle: j= 1, 2, ..., p, 1, 2, ..., p, ...,
${\hat{f}}_{1} ← {s}_{j} \left[{\left\{{y}_{j} - \hat{α} - {\sum }_{k \neq j} {\hat{f}}_{k} \left({x}_{j k}\right)\right\}}_{1}^{N}\right] ,$
${\hat{f}}_{1} ← {\hat{f}}_{j} - \frac{1}{N} {\sum }_{i = 1}^{N} {\hat{f}}_{j} \left({x}_{i j}\right) .$
until the function ${\hat{f}}_{j}$ change less than a prespecified threshold

The data, shown in Table 2, demonstrated that the algorithm identified 381 molecules in the DFG in conformation, 31 molecules in the DFG out conformation and 44 in DFG intermediate conformations.

TABLE 2

| | |
|---|---|
| Total # of molecules | 467 |
| IN conformation | 381 (82%) |
| OUT conformation | 31 (7%) |
| Intermediate conformation | 55 (11%) |
| # of variables | 30 |

The accuracy of the model was determined using the following: [image] sensitivity = # of correctly predicted objects in class1 # of objects in class1 specificity = # of correctly predicted objects in class0 # of objects in class0

Variable selection. Greedy iterative Forward selection based on 70/30 cross-validation analyses; random split into 70% training subset and 30% testing subset; pick the configuration that gives maximum average ROC over a large number (100-1000) random splits.

Merck data: several approaches were attempted: Simple feature selection (e.g., picking top 100 features with maximal correlation, top 100 features maximal variance, etc.), random forest and GAM models. The variable selection did not improve the results compared to random forest. The results with the top 200 variables sorted by correlation are nearly the same as the results when using an entire dataset.

The final results on DFG pattern classification were as follows: Classification of out versus in, inter: ROC =1; classification of in versus inter: ROC=0.991 for example, the threshold 0.5 corresponds to sensitivity= 0.96 and specificity =0.98. The threshold 0.2 corresponds to sensitivity = 0.99 and specificity = 0.995. Classification of activating versus the rest: ROC=0.88. Classification of resistant mutations versus the rest: ROC=0.71.

Analysis of the pocket of the DFG domain was performed. All ligands in the dataset (1900 molecules) were divided into groups of isomorphic molecules. For each receptor in the dataset (1900 receptors), drug molecules and groups if isomorphic drug molecules that physically fit into the receptor’s pocket were determined. The shape of a pocket depends on the drug currently binding to it, so if a drug does not fit into the pocket in its current shape, it does not necessarily mean that the drug cannot fit there in general.

Bias and variance. This represents the way that the algorithm is able to provide the answer to the problem providing statistics and errors distribution. That can be modified to leave the criteria open.

The expected error of a classification (regression) algorithm comes from two sources: 1) Bias the difference between the true value and expected algorithm prediction and 2) variance within the algorithm prediction value:

$$\begin{matrix}\text{Err} \left(\text{x}\right) \text{=} \text{E} {\left(\text{Y-f} \left(\text{x}\right)\right)}^{\text{2}} \text{=} \text{E} \left(\text{Y-\textasciicircum f} \left(\text{x}\right)\right) \text{+} \text{\textasciicircum f} \left(\text{x}\right) \text{-f} \left(\left(\text{x}\right)\right) \\ \text{E} {\left(\text{Y-\textasciicircum f} \left(\text{x}\right)\right)}^{\text{2}} \text{+} \text{E} {\left(\text{f} \left(\text{x}\right) \text{-} \text{\textasciicircum f} \left(\text{x}\right)\right)}^{\text{2}} \text{=} \\ {\text{Bias}}^{\text{2}} \text{+} \text{Variance}\end{matrix}$$

Bagging is a way to reduce the variance by averaging a large number of identical algorithms trained on random subsets of data (example Random forest). Boosting is a way to reduce both by averaging a number of adaptively trained algorithms on different sets of data, such that each next algorithm improves on the objects were previous ones made mistakes (example: AdaBoost). Random forest is simply Bagging applied to the random uncorrelated Decision Trees algorithms. A set of trees are trained on random subsets of data and variables the averaged result from all trees is the final result of a Random Forest algorithm. As an example, generalized Boosting Models is Boosting applied to the Decision Tree algorithm. Decision trees have several properties 1) relatively fast to construct and produce interpretable models; 2) naturally incorporate mixtures of numeric and categorical predictor variables and missing values; 3) invariant under (strictly monotome) transformations of the individual predictors; 4) immune to the effects of predictor outliers; 5) perform internal feature selection as an integral part of the procedure; and 6) resistant, if not completely immune, to the inclusion of many irrelevant predictor variables. Results of the receptor pocket analysis were for the receptor classification problem trained the Random Forest algorithm, received specificity =0.95, sensitivity=0.95. The algorithm out much less weight on individual variables (particularly phosphorylation) and correctly reclassified some of the molecules.

Ligand splitting fragmentation analysis. Splitting the drug molecule and surrounding receptor pocket into distinct functional parts. Spectral clustering algorithm was used in order to split the drug molecules into fragments. The drug molecule graph was used as a similarity graph for the algorithm. We specifically create this task of the algorithm. Any ligand can be divided in small parts until we arrive to a single atom. Similarly, the receptor-binding site can be divided in fragments that interact with the ligand until we arrive to a single atom. Using that option, both the ligand and the receptor were simplified into functional parts related to the interactions between ligand and receptor. Through those well-defined parts “screening” is performed, and similarity is looked for. It is a similar concept to fragment screening (used routinely in some pharmaceutical companies at significant cost and over a long time to obtain the tangible results. The process occurs in seconds of machine time, hence reducing the cost to zero.

### Example III

### Identification of Novel Resistance Mutation in Breast Cancer

Full exon analysis was performed on two patients diagnosed with ER+ breast cancer and who have developed resistance to aromatase inhibitors. Crystallographic analysis of 22,000 gene panels sequenced identified a specific mutation in the tumor cells of both patients that is not present in either patients’ germ line. Patient #1 exhibited a SNP heterologous mutation in the tumor cell in receptor ESR1, Y537S. Patient #2 exhibited a specific isoform of ESR2 receptor with a SNP heterologous mutation, V497M. Further analysis of the ESR1 mutation adjacent to the residue Y537 demonstrated that the sequence clearly identifies the tyrosine kinase phosphorylation site. Any mutation of tyrosine to serine would therefore result in the loss of control of phosphorylation by both the tyrosine and serine kinases. The only possible phosphorylation event that could occur would be the phosphorylation by the dual specificity kinase MEK. The total loss of phosphorylation controls for Patient #2 can be attributed to the deletion of that identical sequence fragment.

The mutations and deletions identified in both patient #1 and #2 suggest that this phosphorylation site plays a critical role in controlling action of ESR1 and ESR2 and therefore forecasts the complete loss of those controlling functions as the resistance to the aromatase inhibitor grows. Both receptors are constitutively active. It was proposed that signaling continues for patient #1 through the MEK kinase and for patient #2 the signal continues through the mutated PIK3CA. Additionally, besides the mutation in ESR2 and the deletion in ESR2, mutation H1047R and in the PIK3CA were identified. Both of these events can result in overriding the effects of initial therapy because ESR1 and ESR2 act Independently on the estrogen receptor and can activate a cancer driven pathway through either the MEK or through the mutated PIK3CA.

A genetic probe was developed that is designed to specifically monitor the presence or absence of the aforementioned segment, 15 amino acids long, to enable an accurate monitoring methodology to detect the earliest signs of a cascading resistance to the aromatase therapy for breast cancer patients that are ER (+). The probe of that specific segment enables the identification of any single point mutation within the length of the sequence. The probe is targeted to Chromosome 6 for the ESR1 receptor and Chromosome 14 for the ESR2 receptor.

The monitoring aspects of the probe require a blood or saliva sample and a sample of the tumor. The difference found between the blood/saliva and the tumor sample is the critical data set. If the probe does not read the sequence of 15 amino acids in the ESR2 receptor sequence located in the chromosome 14, it will mean that the resistance to the aromatase inhibitor is growing and a new therapy should be initiated, Similarly for the ESRI receptor in chromosome 6.

Any single point mutation in the region of the ESR1 and ESR2 receptor is an indication of an increase of activity in the receptor that could develop as resistance to common therapy (including tamoxifen). The different interpretations can help to identify patients for further therapeutic actions based on the type of the resistance. The goal of this genetic probe is to detect the onset of resistance to existing therapy as early as possible. This monitoring provides a significant advantage over simply observing clinical data of the patient suffering the loss of effectiveness in the aromatase therapy and falling into relapse.

### Example IV

### Prediction of Patient Resistance to a Therapeutic Agent

The 3D Pattern Matching Machine Learning Algorithm was used to identify a novel mutation in breast cancer patients. Four breast cancer patients had been given prior targeted therapy and had developed resistance to that therapy. Using the algorithm novel actionable mutations were identified and anti-resistance therapies were predicted for the patients. Further, it was discovered that these novel actionable mutations occur in combination with other known oncogenic mutations and a unique combination therapy was proposed. Additionally, the algorithm predicted the functionality of the novel mutation which was confirmed by the predicted solution. Once the algorithm is provided the full functional genome sequencing of a novel mutation, then the process of structural validation starts. Several tasks are run, and the algorithm will reach a solution based on different variables (one of them is the critical hydrogen bond network in a specific, selected by trained algorithm, regions. The final answer, after comparing several three-dimensional regions, provides the functionality status (activating, resistance or “passenger”) and this directs the therapy including the specificity profile run on the proposed inhibitors to minimize the toxicity profile. The target is either specific gene or pathway. Critically, the algorithm also provides a combination of therapy with a combination tox profile (off target).

### Example V

### Selection of Therapeutic Agents for Specific Mutations

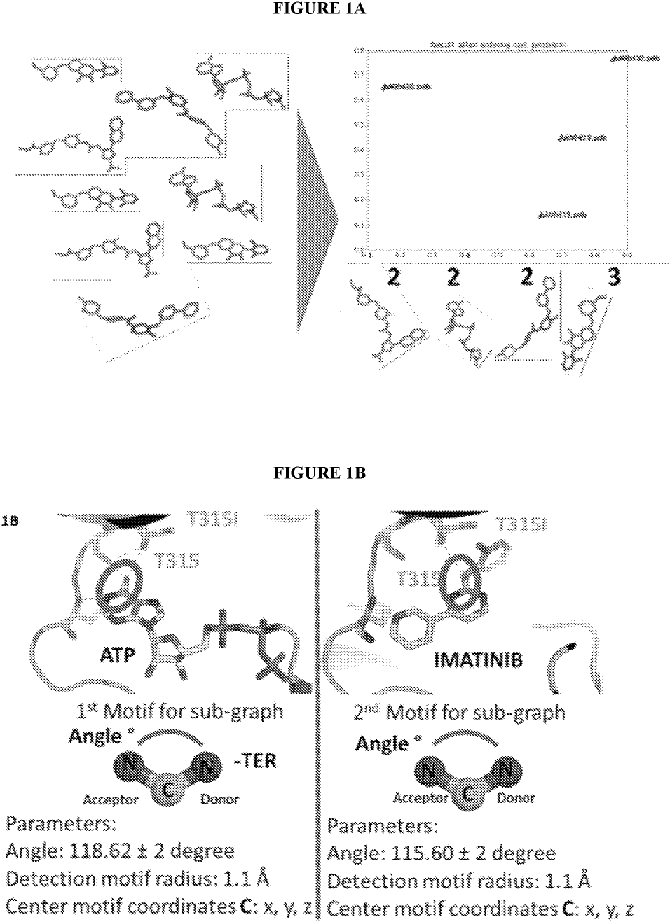

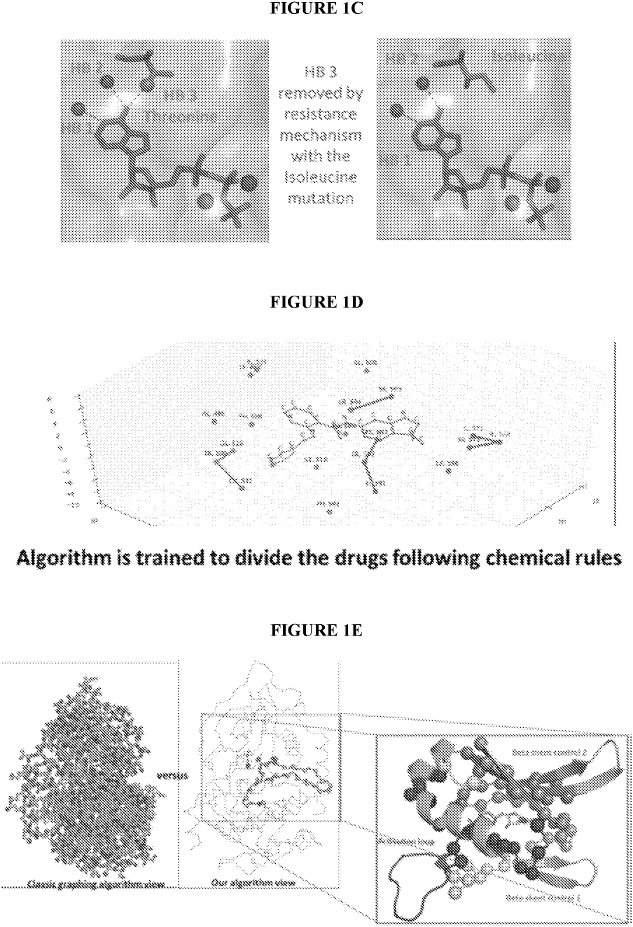

The pattern matching algorithm was used to identify the three-dimensional motif of a chemical scaffold and then used for further modifications required for specific genetic makeup of a patient or group of patients. The algorithm rapidly generated combinatorial modifications to create unique scaffolds (FIG. 1A). The algorithm grouped scaffolds based on their three-dimensional structural patterns. The unique and critical pattern of hydrogen bonding of a small molecule (imatinib) to the “linker” between the upper and lower lobe were detected through three dimensional pattern analyses, including the changes of the hydrogen-bonding pattern due to drug resistance (FIGS. 1B and 1C). Each molecule was subdivided in the three-dimensional space based on the chemical rules in order to determine the “pocket specificity” (FIG. 1D). The algorithm was taught, using the three-dimensional pattern matching, to “walk through” the polypeptide chain toward a specified “specificity pocket” (FIG. 1E) of protein kinase (PKA). Those specificity pockets are very different in the protein kinase DFG motif in conformation (DFG in) vs. the DFG out conformation (DFG out). Using the 3D pattern matching algorithm, it was determined that both activating, and resistance mutations create intermediate states with unique specificities. These states were identified using three-dimensional pattern matching analysis and a protein kinase crystal structure library. Using a crystal structure database of therapeutic agents binding to the target kinase, specific therapeutic agents were selected to target the unique conformations associated with cancer activating or resistance mutations.

### Example VI

### Predicting Conformation of Activating and Drug Resistance Mutants

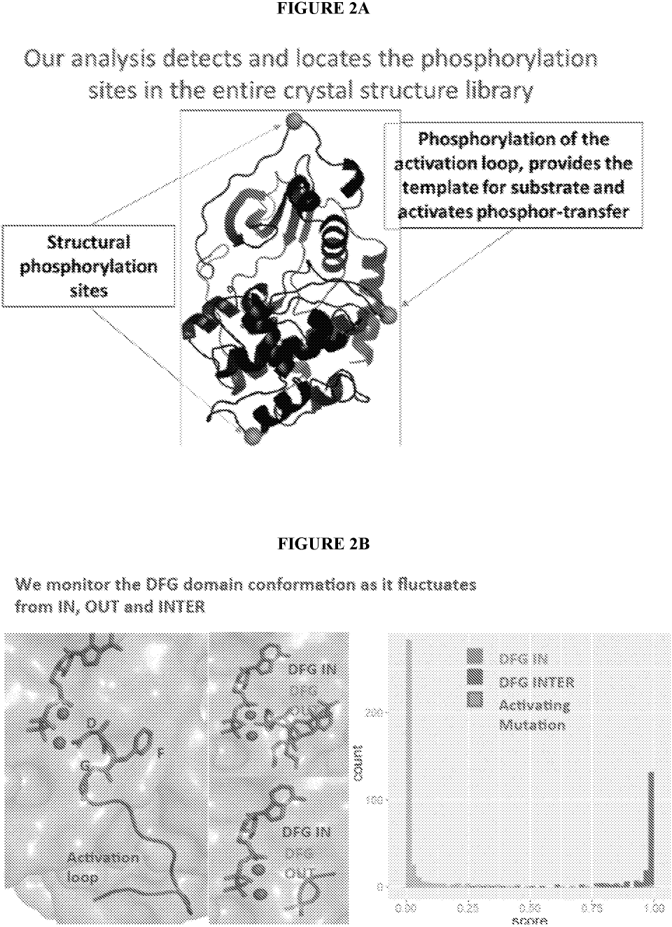

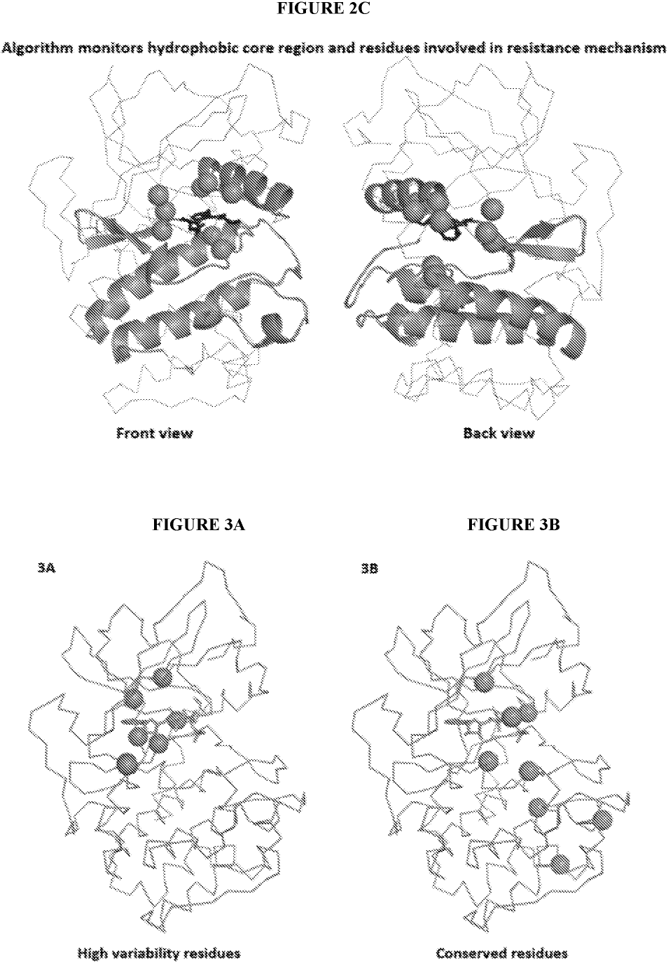

The pattern matching algorithm was used to identify kinase conformation upon phosphorylation and de-phosphorylation. The specificity profile of an inhibitor depends on the conformation of the kinase target. The algorithm recognized the in and out conformation of the DFG motif as well as intermediate conformations (described in previous example). Analysis of one of the intermediate conformations identified one that was associated with activating mutations exclusively positioned on the two pivots of the activation loop and a second group associated with drug resistance mutations forming the hydrophobic core of the most conserved region of the kinase catalytic core. The phosphorylation of the activating loop is a critical part of the activating mechanism of many kinases. The 3D pattern matching algorithm successfully predicted the pattern associated with the phosphorylation of the activation loop (FIG. 2A). Using crystallographic analysis of over 2000 crystal structures, the algorithm predicted the intermediate conformation (FIG. 2B). The hydrophobic network of residues identified by the algorithm and the hydrophobic resistance mutation that keeps the network intact in which neither the in nor out DFG conformation is available. The activation mutation, acting on the two pivots of the activating loop, create an intermediate conformation of the DFG motif (FIG. 2C). The specificity profile of the intermediate conformation is neither in nor out creating the template designing small molecules which target the cancer activating or resistance mutation.

### Example VII

### Predicting the Specificity Profile of Novel Small Molecule Inhibitor

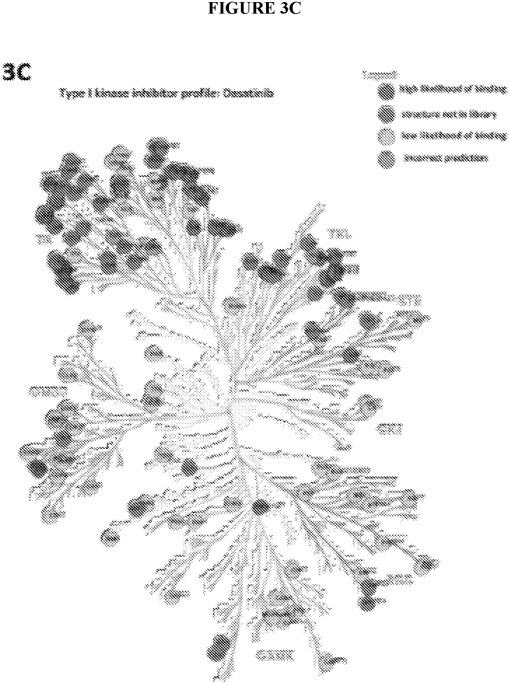

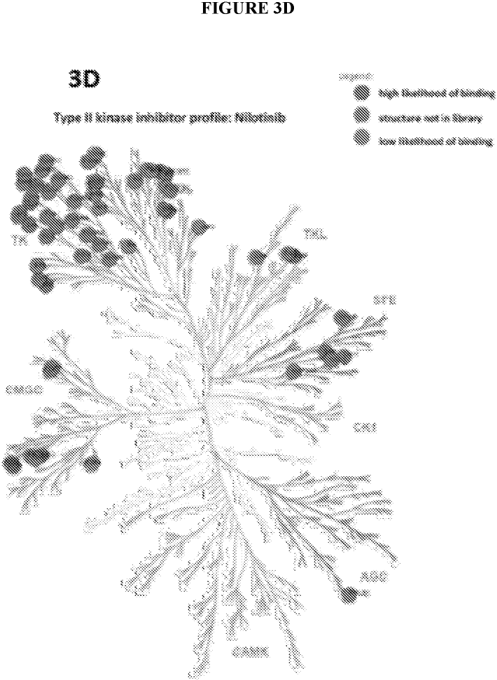

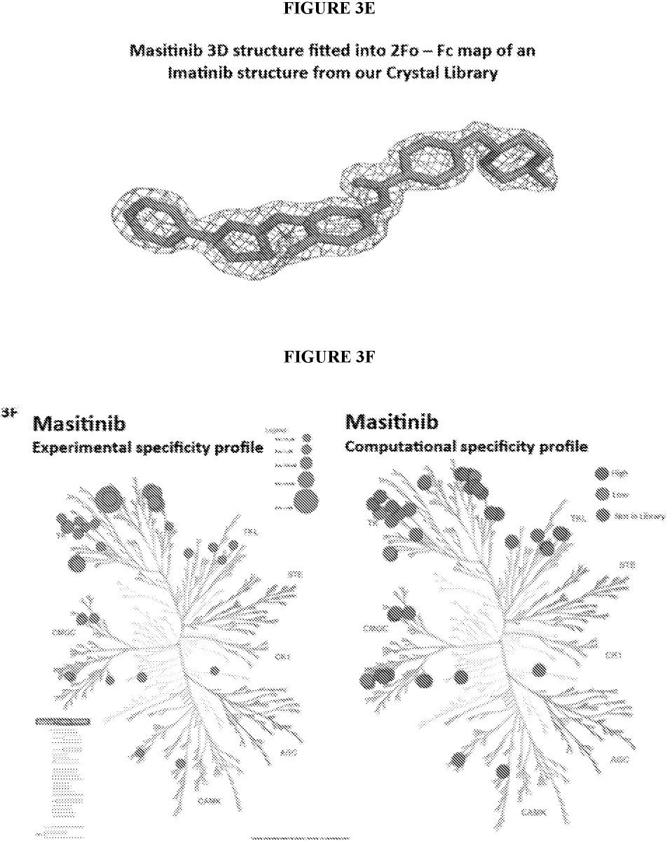

Selection of a scaffold using an algorithm also require selection of specificity profile of the desired scaffold to create a patient molecule or molecules. The need is not simply to define which residues dictate the particular conformational state and which residues do not, but also to define the unique pockets characteristic for the subgroup of the kinases, this grouping is based on the extent of conservation and can be identified the algorithm’s use of the intermediate states. Highly conserved forms a unique pattern of interatomic distances across the two flexible lobes - diverse are pointing out to evolutionary diversity of the ATP binding site. The combination of algorithmic analysis creates the unique pattern for each major conformational state. In those intermediate states associated with activating and resistance mutations the algorithm can predict, even for a never co--crystallized molecule, the correct affinities after a successful prediction of profiles for those molecules, which have been co--crystallized. Predicted specificities for dasatinib (binds the DFG in conformation) and nilotinib (binds the DFG out conformation) resulting from the analysis of the crystal structure library using the 3D pattern matching software were compared to published data. The algorithm correctly predicted the specificity profile for nilotinib and only had three incorrect predictions for dasatinib (FIGS. 3C and 3D). The lower number of targets for nilotinib as compared to dasatinib suggest more “specific DFG out conformation (inactive) than the “less specific” but fully active DFG in conformation. A similar comparison was made for masatinib (FIGS. 3E and 3F).

### Example VIII

### Predicting Activating Mutations

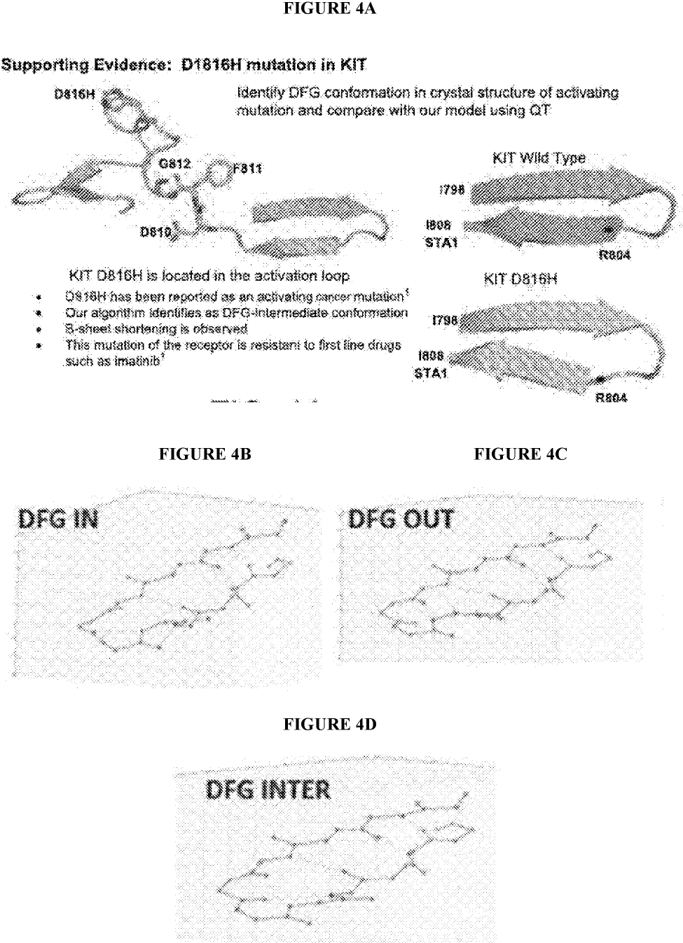

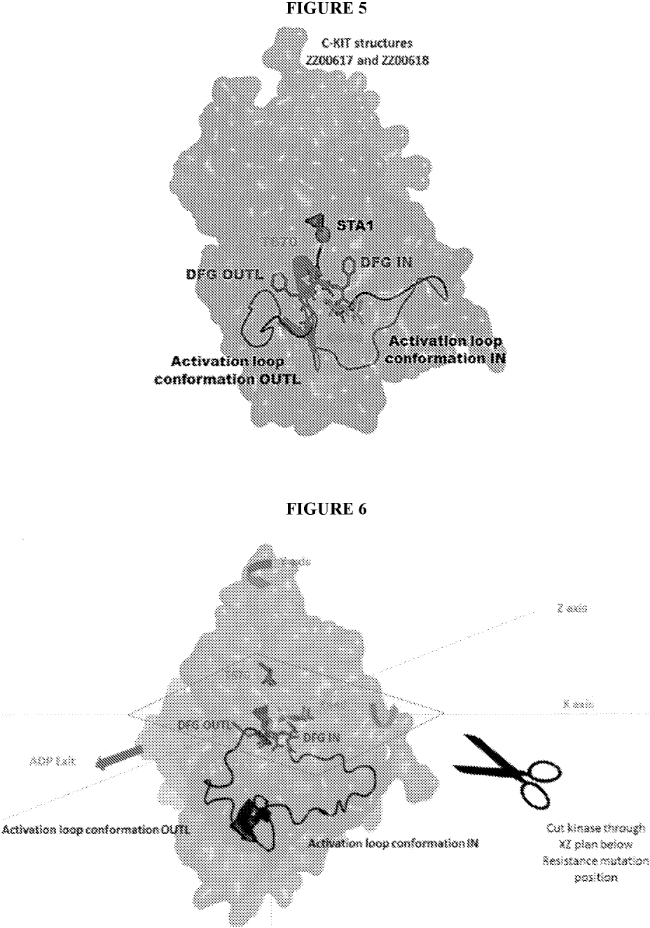

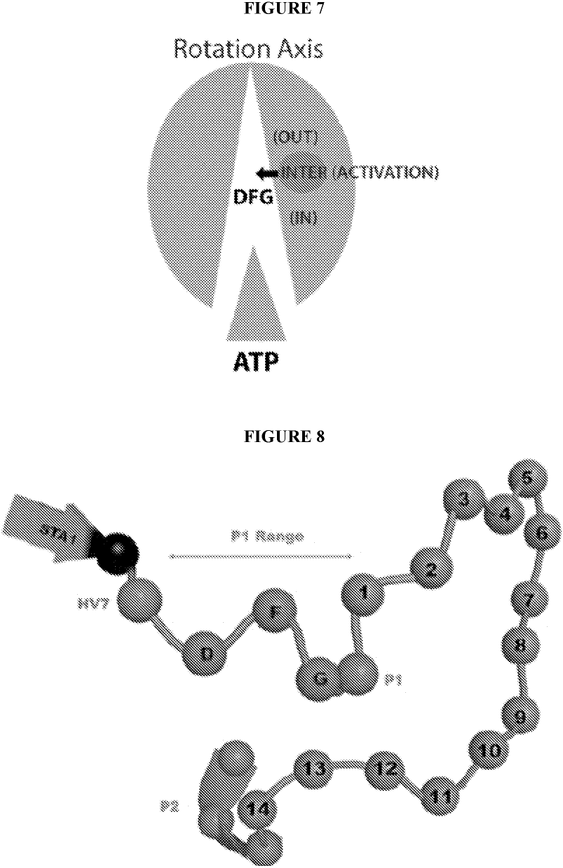

The 3D pattern matching algorithm was used to identify an activating mutation of KIT D816H, which is resistant to treatment with imatinib. The algorithm identified the DFG intermediate conformation associated this mutation (FIG. 4A). Imatinib binds only to the DFG out conformation and does not bind this intermediate conformation. This activating mutation results in shortening of the beta strand and the reorganization of the beta strand hydrogen bonding network (FIGS. 4B and 4C). Since the beta strand lies on the interface between the upper and lower domains of the intermediate conformation of the DFG motif this activating mutation results in changes in the activity of the protein.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding specific embodiments are to be construed as merely illustrative, and not limiting of the scope of the invention in any way whatsoever. The entire disclosure of all applications, patents, publications (including reference manuals) cited above and in the figures, are hereby incorporated in their entirety by reference.

### Example IX

### Molecular Mechanism of Phosphotransfer and Its Regulation in Certain Kinases

The bridge is a kinetic model of the mechanism of phosphotransfer, as deduced from hundreds of somatic mutations of cancer patients, using the three-dimensional pattern matching algorithm and crystal structures library.

The key utilities of this invention emerge from a model that is based on the data of cancer patients.

In essence, the original discovery of the structure of the ternary complex of the catalytic subunit, with Mg ATP and substrate, mimics the specific inhibitor peptide that resulted in the first three-dimensional template used for medicinal chemistry to design Gleevec®.

This template was very successful in part due to the homology modeling conducted by using the coordinates of the aforementioned ternary complex. This revealed amino acid diversity at various pockets around the ATP binding site. The success of Gleevec® in clinics resulted in the unprecedented development of kinase inhibitors. Today there are 33 FDA approved drugs.

However, an alarming, unanticipated consequence emerges over time - patient’s resistance to the drug. The most prominent resistance site is located at the “linker” connecting two lobes of the enzyme. The “Gate keeper residue” term was coined, and numerous publications refer to this site while lacking a clear understanding of what it means in terms of the mechanism of the enzyme.

The forgoing problem was attacked by detailing the molecular mechanism by using hundreds of patients mutants, 7 million unique XYZ kinase coordinates (stored in the crystal structure library), and the proprietary, super-fast three-dimensional pattern matching algorithm (licensed exclusively from first Commercial Quantum Computer Company D-WAVE).

The final results of the calculations were validated using thermal shift calorimetric (TSA) and surface Plasmon resonance (SPR) as applied to the GIST resistant mutant of ckit T670I.

The corollary of this invention, based on the kinetic model of phosphotransfer, is a departure from the original design strategy of Gleevec®. This design strategy is an evolution in that it is a new three-dimensional template that encompasses the pathway wherein cancer “hijacks” the phosphotransfer mechanism in kinases as activated by phosphorylation. In essence, the patient response provides the three-dimensional template for drug design. This evolution is made possible through pattern matching technology.

### Example X

### Hijack Mechanism of Cancer

Over six hundred publications are covered by the kinase somatic mutations library. The mutation selected for further analysis only occurs in the catalytic cores of kinases in cancer patients, and it has been reported in all cancer indications. An aspect of the present invention is the use of the inventors’ crystal structures and algorithm, and the results revealed by the crystal structures and algorithm together with the use of biophysical methods. A further significant aspect of the present invention is the three-dimensional template for the medicinal chemistry focused design.

The known activating and resistance mutation within the catalytic core are outlined in Table 3.

TABLE 3

| | | |
|---|---|---|
| Drug | Activating Mutations | Resistant Mutations |
| Afatinib | EGFR: L858R | EGFR: T790M |
| Axitinib | No Study Found | No Study Found |
| Bosutinib | BCR/ABL: H396P/R, F359V, Q252H and L384M | BCR/ABL: T3151, V299L |
| cabozantinib | MET Y1248H, D1246N, K1262R | VR04M |
| Ceritinib | No study found | ALK: L1196M, G1269A,L1152R, C1156Y, G1202R, and S1206Y |
| Cetuximab | BRAF: V600E | EGFR: S492R |
| Cobimetinib | Braf: V600 mutation | No Study Found (newer drug) |
| Crizotinib | ALK: I1171N R1275Q | ALK: S1206Y, L1196M, G1202R |
| Dasatinib | BCR/ABL: H396P/R, F359V | BCR/ABL: T315I |
| Erlotinib | EGFR: L858R | T790M |
| Gefitinib | EGFR: L858R, L861Q | T790M |
| Ibrutinib | BTK: L265P | BTK: C481S PLCy2: S707Y, R665W, and L845F |
| Imatinib | No study found | KIT: T670I ABL: T315I |
| Lapatinib | HER2: G309A, D769H, D769Y, V777L, V842I, and R896C | HER2: L755S |
| Lenvatinib | RET: C634W, M918T | No Study Found (newer drug) |
| Nilotinib | BCR/ABL: F317L | BCR/ABL: T315I, E255V |
| Osimertinib | EGFR: T790M | No study found (newer drug) |
| Palbociclib | ER-LBD: Y537S, Y537N and D538G | No Study Found |
| Pazopanib | No Study Found | No Study Found |
| Pegaptanib | No Study Found | No Study Found |
| ponatinib | T315I mutant ABL | BCR-ABL 1T3151/F359V |
| Regorafenib | No Study Found | No Study Found |
| Ruxolitinib | JAK2: V617F | JAK2: V617F |
| Sorafenib | PDGFRA: T674I | FIPILI-PDGFRA: D842V |
| Sunitinib | KIT: L576P | KIT: D816H/V |

The resistance mutations resulting from the action of specific drugs within the catalytic core are outlined in Table 4.

TABLE 4

| | | | |
|---|---|---|---|
| Gene | Mutation | Drug | Finding |
| ABL1 | T315I | Imatinib | Patients were shown to have the mutation both before and after drug use. |
| EGFR | T790M | Gefetinib/Erlotinib | Mutation acquired via the drugs. Mutation not shown pretreatment. |
| ERBB2 | T798I | Lapatinib | Not reported to be in a before/after study but does cause drug resistance. |
| KIT | T670I | imatinib | not found in tumor samples prior to drug testing/found to cause drug resistance. |
| PDGFRA | T674I | imatinib | Multiple papers refer to it as an acquired mutation. This seems widely accepted. |

It is important to refer to clinical data that clearly indicates in some cases, the “resistant” mutation has been present prior to administering the drug. As has been presented in Tables 3 and 4, the specific cancer indications are not listed, but this data is used for interpretation of the clinical results based on the three-dimensional network of interactions between the mutated cancer patient target and a specific drug. Perhaps, the most intriguing part of the initial analysis is that the clinical results suggest that patient response can be translated into three-dimensional space (crystallographic analysis with pattern matching algorithm) and that the resulting analysis can be used to discover the intimate mechanism regulating the activity of the patient’s kinase target.

The patient’s kinase regulatory domain mutants cannot be translated, but, through pattern matching analysis, both the activating and resistance mutations can be correlated with the binding of the drug, and more importantly, how the binding of the ATP competitive inhibitor results in aggressive resistance, and subsequently, how this knowledge can be used for design based on the genomic information of the patient. Here the predictive methodology has been applied to two critical 3D patterns; the very highly conserved DFG motif, and the highly variable linker region. Both have been originally described in the crystal structure of PKA.

### Example XI

### Identification of Phosphate Conformation

The original 3D pattern matching algorithm described herein constitutes a crucial tool as an algorithmic phosphate detector to rapidly identify phosphate on the activation loop (see FIG. 2A, and Example VI). The algorithmic detector allows the algorithmic identification of activating phosphate in DFG IN, DFG OUT and DFG INER conformations; which provides clues that enable a clear distinction as to whether a kinase is active or inactive.

244 crystal complexes were identified, with oncogene kinase with small molecules capturing the DFG INTER state of that oncogene, and the absence of an activating phosphate; this large pool of data suggests that the gene fusion mechanism of that oncogene creates a novel DFG INTER state, which is very active without an activating phosphate. Hence, it provides algorithmic evidence that is based on precise data of decoupling the control in this oncogene. Therefore, demonstrating that the phosphate detector is crucial as a tool to identify phosphate in IN/OUT/INTER conformations.

Target validation, relies on detecting or not detecting the phosphate on the activation loop of DFG INTER conformation, based on the determination of the conformation, by using the “algorithmic phosphate detector” in combination with DFG INTER conformation detector. This provided, for the first time, a clear molecular definition of “hijack” by a cancer activation event of the structural mechanism of the kinome core signaling. It provided over-whelming; statistically significant structural evidence derived by our methodology of loss of control of the activity of the kinase core if the core is activated by cancer.

In DFG INTER conformation the activation loop is un-phosphorylated, yet the oncogene is extremely active. There is no better example of a structural mechanism identified by A/I in a large number of oncogenes to show where lies the origin of the cancer driven loss of signaling control.

Hence, both detectors, if combined together, provide identification of the real target for kinase inhibitor design. That is the oncogene in DFG INTER conformation with un-phosphorylated activation loop.

### Example XII

### Designing Chemistry of New Scaffolds Drug

The initial discovery of the specific region where amino acids are being exchange within the evolutionary tree of the kinome suggests the positions where the combinatorial power of evolution is the highest and creates a unique pattern for each kinase and might be used for the drug resistance mechanism for molecules that are actually designed to target those sites “specificity pockets” (high diversity - high potential of pocket of specificity).

This illustrates a very novel approach of designing the chemistry of new scaffolds to address cancer patients drug resistance to FDA approved kinase inhibitors (see FIG. 3A and Example VII). This approach is based on treated patient genetic responses and enables to deliver structural proofs.

These proofs lead to the logical creation of a three dimensional “surface net” of DFG INTER conformations of the selected oncogene. This “INTER-NET” allows to screen small fragments, from a proprietary library of fragments that capture and bind into the target, which is the DFG INTER conformation of selected oncogene protein. This created INTER-NET is actually an algorithmic “net” of distances in 3 dimensions to “catch” the specific molecules binding exclusively to the DFG INTER conformation of the selected oncogene -this INTER-NET will be expanded for other oncogenes using the same principles (see FIG. 1D and Example V). The algorithmic “training” can be used to divide the oncology targeted drug by following chemical rules.

The algorithmic fragmentation of the scaffold structure is useful (using our algorithm) and it will be used going forward in building a drug from the DNA SEQ’s INTER fragments. First, having an oncogene DFG INTER NET, the best anti-resistance FDA approved drug structure can be deconstructed, and secondly reconstructed from the oncogenic DFG INTER library to construct the new drug scaffold. In both cases specific oncogene DFG INTER-NET (NET is from “fishing net”) will be used, as defined by the surface of oncogene protein.

The discovery of the “fishing net” relies on several tenets.

Tenet one: the kinome evolutionary process of adaptation of ATP binding cleft results in the seven high amino acid diversity (HVR) regions.

Tenet two: the rotational mechanism of kinome signaling with specific Y axis comprises crucial INTERMEDIATE state.

Tenet three: the INTERMEDIATE state is captured by patient cancer activating mutation

Tenet four: capturing the rotational mechanism results in a unique distance shortening between the pivots of Y axis of rotation in the INTER state of the oncogene.

Tenet five: the 1 Å shorter axis of rotation in the INTER state of oncogene results in changes of ATP/ADP kinetics

Tenet six: none of the FDA approved drugs bind onto the oncogenes’ INTERMEDIATE states.

Tenet seven: as a result, targeted oncology kinase inhibition suffers persistent drug resistance.

Tenet eight: the highest frequency of drug resistance mutations occurs within the HVR region at the HVR4 pivot of the axis of rotation.

Tenet nine: there are two pivots of axis of rotation: HVR4 and HVR7. The 1 Å GAP (or shortening) is between HVR4 and HVR7 pivots .

Tenet ten: every kinase consists of a “water tunnel” filled up with crystal waters. The roles of those waters are crucial, as the axis of rotation of the two domains during phosphotransfer protrude through this water tunnel. Hence the water structure defines the kinetics of rotation and defines the kinetics of phospho-transfer. Rotation of one domain vs another is associated with the displacement of thousands of atoms of one domain vs thousands of atoms of another domain. Some displacements account for several angstroms. Some displacements are not detectable by standard X-ray techniques.

Tenet eleven: in DFG INTER conformation of oncogene, with ATP analog bound, the 1 Å shortening of the Y axis results in the re- arrangement of the crystal water molecules leading to changes in the ATP/ADP kinetics. Onco-kinase now is signaling differently among a vast cellular network.

Tenet twelve: the diversity of the water structure kinome networks is driven by the unique diversity of amino acids of every kinase which constitute two pivots: HVR4 & HVR7. Changes through drug resistance mutation occur, as a result of the action of drugs, and results again in changes in the kinetics of cancer patient signaling kinase target.

Tenet thirteen: all twelve previous tenets constitute the experimental frame for the three dimensional design of INTER net or “fishing net” to screen fragments, leads, IND molecules and drugs to select the best molecule to by-pass, in early screening, the critical common mechanism of kinome signaling that is hijacked by the activating genetic event.

Our recent virtual screening using the INTER net or “fishing net” resulted in the rejection of most of the 20 FDA approved drugs. The rejection based on their structural ability that was derived by the chemical structure to enter the drug tunnel and thus enhance cancer patients drug resistance. Only two drugs which has been screened by INTER- net did not enter the water tunnel and were selected by the “fishing net” to proceed forward to algorithmic fragmentation to create the best anti-resistance scaffolds. This step wise design is selecting analogs from catalogs, which are then “refined” by med chem which is guided closely by algorithmic specificity analysis.

“Fishing”, using the “INTER net” screening will be carried out fast and continuously through the entire repertoire of all FDA kinase inhibitors drugs until all candidates for fragmentation for all cancer indications are selected.

The following FDA approved inhibitors have been screened: dasatinib, imatinib, nilotinib, bosutinib, regorafenib, sorafenib, ponatinib, sunitinib, vermurafenib, vandetanib, ibrutinib, abemaciclib, ribociclib, palbociclib, axitinib, crizotinib, gilteritinib, erlotinib, midstaurin, ruxolitinib, brigatinib, osimeritinib.

Validating the robustness of the “fishing net” within a few days of introduction suggests that a final set of methods to carry fragmentation of FDA approved drugs, to select fragments for a fast run of limited med chem, towards the creation of a novel platform of kinase inhibitors with a totally different mode of binding have been established. As a proof-of-concept, the best design kinase inhibitor on the market was selected (the best criteria were clear: drug with minimal patient drug resistance); with just a few minutes run of the “fishing net”, using over 20 FDA approved inhibitors, a single molecule was identified; and it was the molecule initially selected.

### Example XIII

### Material and Methods for Examples XIV-XV

The “family” of the human kinome encompasses over 500 members of signaling molecules and the homologous catalytic core consists of several invariant amino acid’s residues, one of them is pattern DFG. The DFG pattern is an integral part of the activation loop of the catalytic core. The activation loop is subject to “activation” through phosphorylation and “deactivation” by dephosphorylation. Synchronization of the DFG conformation with the phosphorylation of the activation loop is the essence of intrasteric regulation of the kinome. (Knighton et al). The activation loop forms the “saddle” for the protein target to be phosphorylated. The DFG IN conformation is the active phosphorylated conformation when the protein is “open” for interactions with a specific group of drug molecules. The OUT unphosphorylated conformation is, in contrast, an inactive conformation wherein the protein is “open” for engagement with a different group of drugs. The general classification of inhibitors of catalytic core falls into those two categories.

Two other conformations have been discovered using 1624 structure of kinases and using the pattern matching and machine learning algorithms. A kinase with this DFG INTER conformation is an unphosphorylated conformation wherein the protein can interact with the drug, and the activation loop has a different conformation from the activation loop with a DFG IN conformation. The second conformation discovered is the OUTL conformation. In this conformation, the DFG pattern is as in the OUT class, and D still aims in the ATP binding region. In OUTL conformation of F of DFG completely overlaps the ATP binding region and has a direct pi-pi interaction with the drug, or ligand, bound to the kinase. F of OUTL has a position mirrored to the F in the IN conformation.

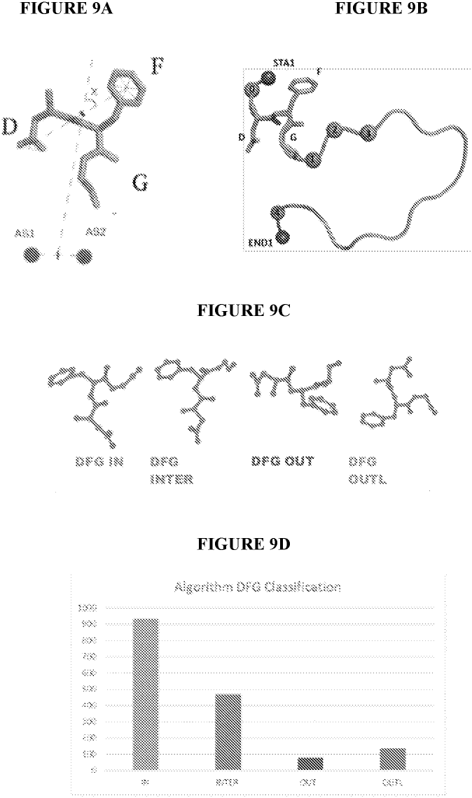

Hence these conformations can be classified into four general groups: IN, INTER (short for intermediate), OUT and OUTL (see FIG. 9A). One of the objectives of this work was to develop an automated procedure to detect these four types of DFG pattern conformation to then be able to screen large databases of existing molecules. There is, however, a considerable variability in spatial conformations within each group, as no two molecules have an identical spatial structure. Similar to how there is a variability in images of cats and dogs and there is no simple set of computational rules that would be able to distinguish those two classes, there was no evident set of criteria that would reliably separate the DFG conformation groups. Instead of trying to build such a set of rules manually and inevitably introduce errors and biases, to instead learn the separation model from real data using a machine learning approach was chosen.

Machine learning algorithms are able to learn complex patterns and decision criteria by utilizing large amounts of real data and gradually tuning parameters in powerful computational models (possibly references to some examples/books/papers). They have been successfully applied in a number of domains, including drug discovery (references here, e.g. AlphaFold https://rdcu.be/b0mtx).

To train a DFG conformation classification model, a random forest algorithm developed by Breiman et al., 2001 was used [Breiman, L. (2001). Random forests. Machine learning, 45(1), 5-32.]. Random forest algorithm exhibits several attractive properties, such as computational efficiency, interpretability of resulting models, resistance to the effects of predictor outliers and inclusion of irrelevant predictor variables, etc. [James, G., Witten, D., Hastie, T., & Tibshirani, R. (2013). An introduction to statistical learning (Vol. 112, p. 18). New York: springer.] It was found that this algorithm generalized better to the unseen data then other machine learning algorithms. In order to train the model, 600 molecules from out data set into four classes based on DFG were manually labeled conformation: IN, INTER, OUT and OUT-L. This data was then split into 70% training and 30% validation sets. The model was trained on a training set, and the classification results were evaluated on a validation set.

In order to improve learning performance, a feature engineering step was conducted, where close to 40 variables that intuitively appeared to be predictive of the DFG conformation class and represented each molecule with the corresponding values of these variables were designed. Most of these variables represented geometrical features, such as distance from the D pattern to the activation sites, which is a good discriminator between IN and OUT classes. During training, the algorithm learned to integrate the information from these 40 variables into a more accurate classification criterion.

The resulting prediction accuracy for each class in presented in Table 5.

TABLE 5

| Class | Accuracy |
|---|---|
| IN | 0.96 |
| INTER | 0.93 |
| OUT | 1 |
| OUT-L | 1 |

The algorithm achieved a high accuracy in predicting all four classes. As expected, the largest ambiguity was between IN and INTER classes, was a small percentage of the molecules were incorrectly classified to a wrong class. OUT and OUT-L classes, on the other hand, were classified perfectly on the validation data, likely because these conformations are very different from other classes in terms of geometrical structure, and it is possible to discriminate them unambiguously.

A trained model was then applied to all the molecules in our dataset, resulting in the predicted distribution shown on FIG. 9D.

### Protein Expression and Purification

For proof of concept of identifying drug candidates which binds to T315I mutant, this mutant (residues 229-499) was expressed in a bacterial vector that with a 10X His tag; groEL was also co- expressed to facilitate proper folding of soluble expressed abl T315I. Additionally the vector also expressed the phosphatase yopH to yield unphosphorylated abl T135I; lack of phosphorylated protein was verified by LC/MS. His tagged and His tag removed by TEV protease was verified by sequencing and thermal stability.

ABL1(T315I) (isoform la) residues 229-499 with an N-terminal 10xHis tag has been co-expressed with recombinant YopH phosphatase. The protein will be referred to as 10xHis-tev-ABLl(T315I)(229-499) in this manuscript. Biomass production and protein purification are based on Albanese et al. (2018, Biochemistry 57, 4675-4689) with minor modifications. Briefly, the protein purification consisted of TALON affinity chromatography. The resulting protein was stored at -80C after flash-freeze in liquid nitrogen. In three purification attempts we have changed the following parameters: The expression temperature was reduced from 30 C to 18 C and apGro7 plasmid was added with groES-groEL on it. This resulted in better expression of ABL1 but more biomass (11 L) was needed in order to add additional purification steps (ion exchange chromatography and size exclusion chromatography). The overall amount of purified protein was increased 3-4-fold, clearly, the addition of groES-groEL was beneficial. The final protein phosphorylation state was established by Western blot using anti- phospho-Tyr393 Ab (Abeam ab4717). ABL1 is completely dephosphorylated. Furthermore, ABL1 is active and can be auto phosphorylated if ATP/MgCl2 is added.

### Chemical Analogs Binding to ABL1 (T315I) Using eSPR

Surface plasmon resonance (eSPR)requires the attachment of the ligand to a surface and then flowing the analytes across this surface to measure the kinetic rate of the association (ka) and the dissociation (kd) of the ligand with the protein. From this measurement the equilibrium binding constant (KD) is calculated.

### Example XIV

### Virtual Sorting and “Fishing Net”

For the ligand selection of scaffolds that are prone to bind to the T315I mutant in the DFG INTER conformation, we have designed a novel virtual sort, which we refer to as a computational “fishing net”. Using the mathematical function that was derived by a machine learning analysis of DNA SEQ’s crystal structures library, we identified the XXX (provide number how many) DFG INTER conformations of ligand-kinase co-crystal structures already assembled in our crystal library. A further reduction of binders to (provide number) of DFG INTER conformations as accomplished by including only the binders with the lA shortening of the rotation axis between HVR4 and HVR7.

The process steps and tools leading to the usage of the “fishing net” are listed in Table 2. The operation creating a three-dimensional cluster network consists of the C alpha of HVR1-7 coordinates and the network of distances among those absolutely unique in kinases combination of seven amino acids. At HVR 4 position the frequency of drug resistance mutation in imatinib treated cancer patients is the highest. We explicitly eliminated all DFG INTER binders which protrude HVR17 three-dimensional network “cage”. Within this group, binders with primary amine located at the hedge of HVR1-7 network were include. Cut off molecular weight was 400.

Once identified DFG INTER binders which satisfy our “fishing net” conditions, the chemical scaffold was further minimize using the adenosyl ring of ATP as a model. From that common scaffold we defined two derivatization constituents: R1 and R2. The R1 derivatization is aimed to improve solubility, minimize toxicity and ADME. The R2 is crucial to improve conformational specificity and maximize affinity for DFG INTER. Rotating amino acid during the transition from DFG OUT through DFG INTER into DFG IN is among many, our primary conformational specificity target.

TABLE 6

Lists of the processes, steps, and tools that led us to the usage of the “fishing net”

| | |
|---|---|
| Characteristics | Descriptions |
| ONCOGENE | Selected oncogene is ABL1 mutant T315I and frequency of occurrences of drug resistance mutations in HVR1-7 region is based on 1127 imatinib treated patients. |
| PROCESS | Consists of 3D pattern matching of specific region of kinase oncogene with structure of chemical fragment selected from DNA SEQ INTER fragment library and commercial analog catalogs. |
| STEPS | Virtual sorting of potent analogs as the precursors for drug candidates consists of two steps. Step number one is the crystallographic identification of unique INTER state of targeted drug resistance oncogene. Second is the identification of specificity constituents of the INTER state of that drug resistant oncogene. |
| TOOLS | 3D pattern matching and machine learning algorithms (both pattern matching and machine learning algorithms has been licensed exclusively from quantum chip Company, D-WAVE), over 60 million atoms DNA SEQ kinase crystal 3D structure library with its unique origin and geometry (US Patent & US Patent) and DNA SEQ 3D library of small chemical fragments bound in 214 crystal structure of kinases in the INTER state. |
| VIRTUAL “FISHING NET” | Fishing net is the crucial filtering of analogs from commercial analog libraries. Process is constrained in 3D by the size of “net” permitting |
| | for selection of analogs. Net is defined in such a way that HVR1-7 region is excluded as it is the region of high frequency of drug resistance mutation. |

Each of the scaffolds was used as a primary query in search of the public chemical databases (PubChem, ZINC). For this search, positions R1 and R2 were left open to generate a combinatorial array of analogs. Additional critical queries, like 5 Lipinski’s Rule of 5, solubility, and commercial availability were imposed. This “virtual sort” could still generate thousands of analogs.

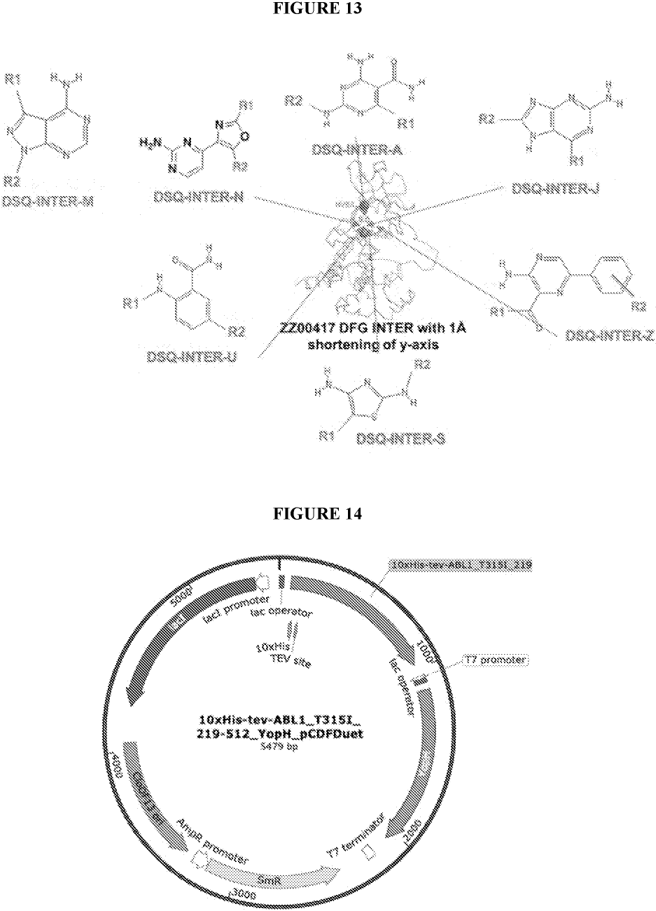

In order to “trim down” the number of analogs to circa 30 analogs per scaffold an extra step was added. The 3D models of the “sorted” analogs was superposed to co-crystal structures of the specific scaffold co-crystallized with protein in the DFG INTER conformations. Several ligands that satisfied both the fishing net conditions and the query search failed to succeed this last step of virtual sorting and were therefore excluded. The final step led us to have one hundred and thirty analogs among all six scaffolds. FIG. 13 provides the chemical structure of selected scaffolds. Each of the scaffolds was used as a primary query to the public chemical (PubChem, ZINC) leaving open R1 and R2 to generate a combinatorial array of analogs. Additional critical queries, like Lipinski’s rule of 5, solubility, and commercial availability were imposed.

The sort could still generate thousands of analogs. In addition to “trimming down” (to circa 30 analogs per scaffold) we have superposed the 3D models of sorted out analogs to co-crystal structures of specific scaffold co-crystallized with protein in DFG INTER conformations. Several ligands which satisfy fishing net conditions and query search failed to succeed this last step of virtual sorting. The final step led us to have one hundred and thirty analogs among all six scaffolds.

The two protein constructs were immobilized on a biosensor chip (through tags, amine coupling, or biotinylation based upon the proteins supplied). Upon attachment of the proteins to the surface, experiments with known binders (ATP, and a standard inhibitor) has been performed to confirm that the attached protein retained this ability to be active. The panel test compounds has been evaluated using a serial dilution and replicates. The kinetic interaction parameters ka, kd, and equilibrium constant, KD, are determined for the test compounds. Unphosphorylated and phosphorylated forms of his-tagged ABL1(T315I) were attached to a Ni- NTA SPR biosensor.

Compounds were evaluated, with fast on-off binding observed for the positive controls Dasatinib and Imatinib. The IC50 values for Dasatinib and Imatinib against the T3151 mutant reported by Chan et.al., [Cancer Cell. 2011 April 12; 19(4): 556-568.doi: 10.1016/j.ccr.2011.03.003] are > 10 µM. KD values estimated by SPR in the current assay are > 100 µM and appear to align with the reported literature. Compounds 172889-26-8, 172889-27-9 and 220036-08-8 bind to both forms of the protein with fast-on-off kinetics.

An algorithm was developed to scan 1614 kinases co-crystal structures of TKs present in public protein data bank (PDB) using 3D pattern matching- and machine learning algorithms licensed from DNA Wave. Of these structures 933 were “DFG IN” and 215 were “DFG OUT” structures. We identified a novel kinase “DFG INTER” conformation for 466 crystal structures (FIG. 10).

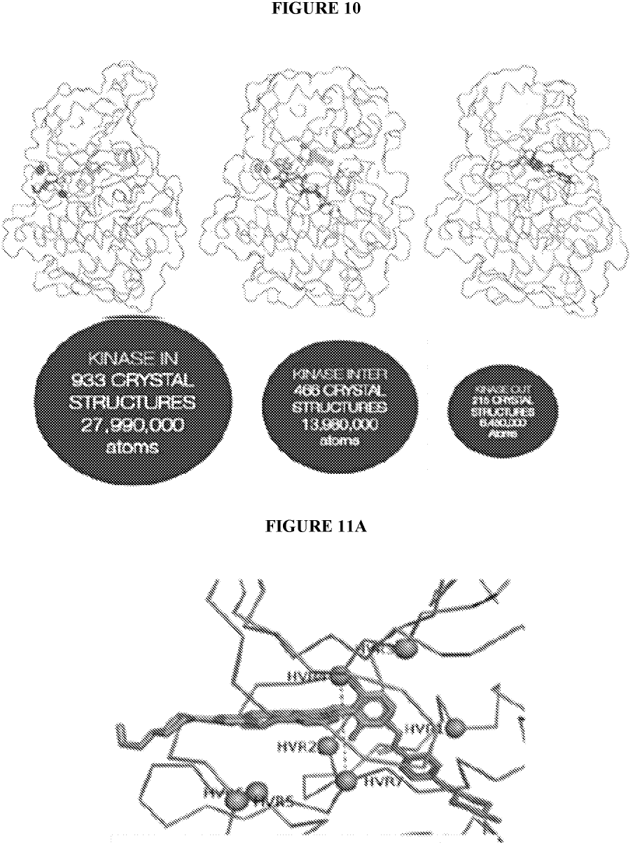

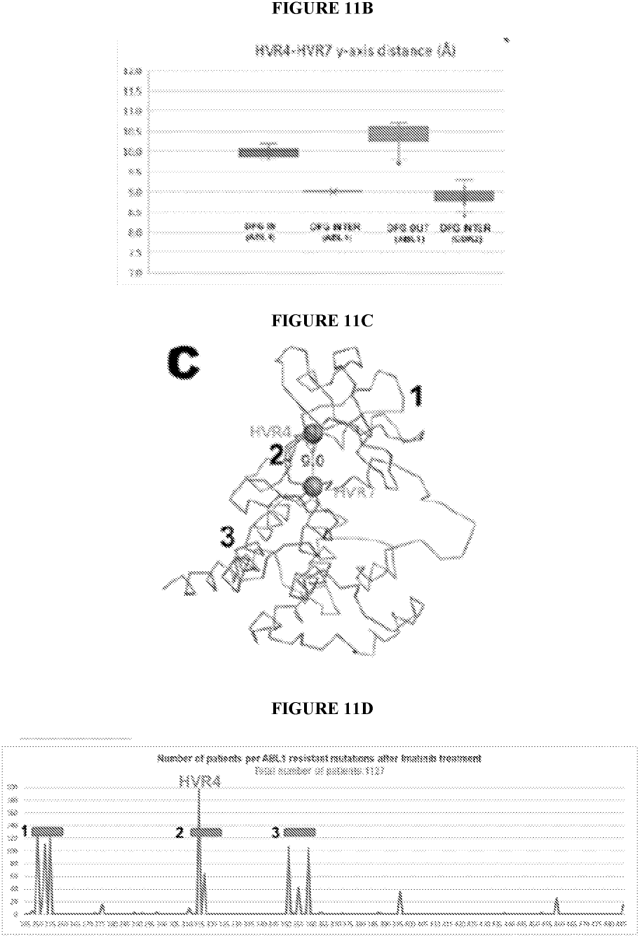

None of available FDA approved drugs that co-crystallize with TKI’s bind with high affinity to the DFG INTER conformation that is essential for TK signaling. Each TK has a unique High Variability region (HVR) in the ATP binding cleft which includes the rotational axis essential for TK activity (FIGS. 11A and 11B) TKs have a unique combination of 7 amino acids. The INTER state differs by the distance along the common rotational axis between 2 pivots: HVR 4 and HVR7. A DFG INTER with 1 Å gap divides oncogenic and cell cycle control kinases from non- oncogenic kinases. Overall Kinases DFG INTER consists of 2 subgroups: one with a gap (220) and the other without a gap (240).

Next, the number of patients with single drug resistant mutations within the network of HVRs was analyzed. The common drug resistant mutations with the highest frequency occur on one pivot of the rotational axis of TK activity. The HVR region with the 1 Å gap is the most common drug resistant mutant (FIGS. 11C and 11D). Review of the literature reveals that abl T315I is the most common drug mutant in CML (ref; Drucker may want to write insert here).

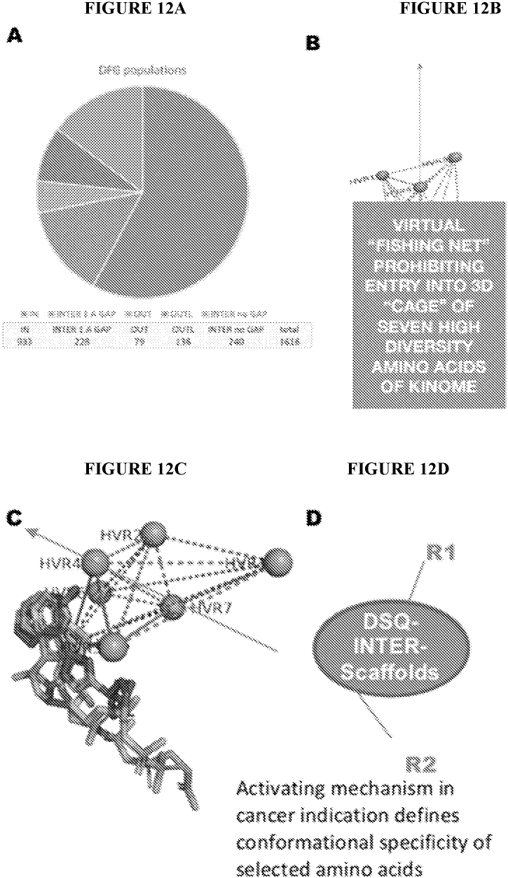

Using this process of “fishing net” sorting, we generated six scaffolds called DSQ-INTER- J, A, N, U, S, Z and additional four scaffolds in “reserves” to replace any original scaffold which could fail for any reason during experimental validation (see FIGS. 12A-12C and 13). The six primary scaffolds were used to generate a series of analogs to be tested using eSPR and after further selection to validate them in vitro using treated CMI, cancer patient cell lines (see below).

### Example XV

### Generation and Characterization of a Mutant ABL1(T315I) Hinase Domain

The goal of these experiments was to produce some tag-removed ABL1(T315I)(229-499) for crystallographic studies. In addition, protein was also produced during the course of the purifications that retained the lOxHis-tag to enable studies by Surface Plasmon Resonance. The recombinant ABL1(T315I) (isoform la) residues 229-499 protein was designed with an N-terminal 10xHis tag with a TEV protease cleavage sequence and was co-expressed with recombinant YopH phosphatase (to maintain the ABL1(T315I)(229-499) dephosphorylated form). The protein was also co-expressed with the pGro7 chaperone to improve the level of soluble protein expression. Biomass production and protein purification are based on Albanese et al. (2018, Biochemistry 57, 4675-4689) and Wilson et al. (2015, Science 347, 882-886) with minor modifications. Briefly, the protein purification consisted of TALON affinity chromatography, TEV cleavage, Ni-NTA, ion exchange chromatography and SEC. The resulting protein was concentrated and stored at -80° C. after flash-freeze in liquid nitrogen.

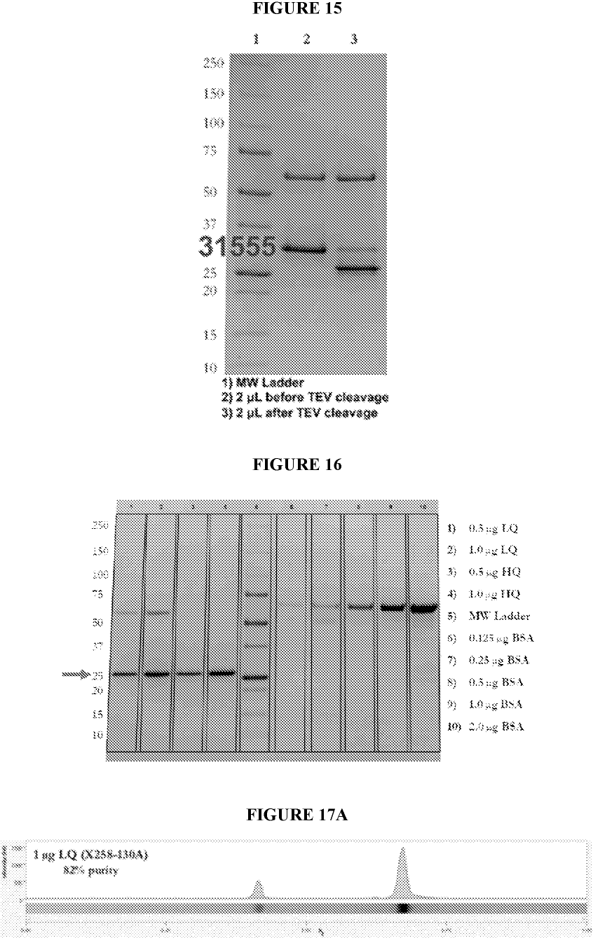

The ABL1(229-499)(T315I) kinase domain protein was produced through E. coli expression, with co-expressed with YopH phosphatase (to maintain dephosphorylated state) and a chaperone protein (to facilitate soluble expression) (see FIG. 14). As illustrated in FIG. 15, TEV cleavage was efficient at removing the His tag.

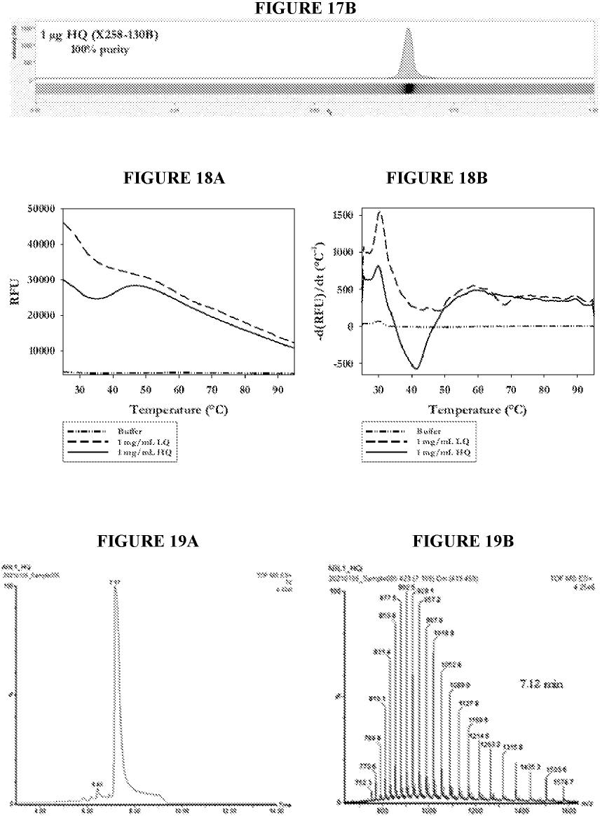

A total of five rounds of protein expression and purification have been undertaken to optimize the methods and generate mg quantities of highly pure ABL1(229-499)(T315I) protein, both in the unphosphorylated and as the phosphorylated form. This provided the means to optimize the various stages of the process and to increase yields. The last round of purification involved 11 Liters of cell culture with and overnight induction at 18° C. As illustrated in FIG. 16, the purification of the kinase domains was assessed in a Coomassie 4-20% gradient gel in reducing conditions. In lanes 1 and 2 partially purified ABL 1 (T315)(229-449), LQ (lower quality) was obtained by removing the tag and was found 82% pure by densitometry (see FIG. 17A), as it retained some of the pGro7 chaperone. The 3.8 mg amount was stored for future repurification. As shown in lanes 3 and 4 high purity unphosphorylated ABL1(T315)(229-449), HQ (high quality) was obtained by removing the tag and was found 100% pure by densitometry (see FIG. 17B). A total of 6.0 mg of pure protein was stored in aliquots for using in the biophysical studies. Yield of 0.55 mg/L culture. Lanes 6-10 includes BSA standards.

As shown in Table 7, and in FIGS. 18A-18B, the purity of the protein impacted the melting temperature.

TABLE 7

Melting temperatures of tag removed ABL1 (T315I) (229-499).

| Construct | Tm (°C) |
|---|---|
| Buffer | / |
| LQ: 1 mg/mL | very weak signal, 42-48 |
| HQ: 1 mg/mL | 41.5 |

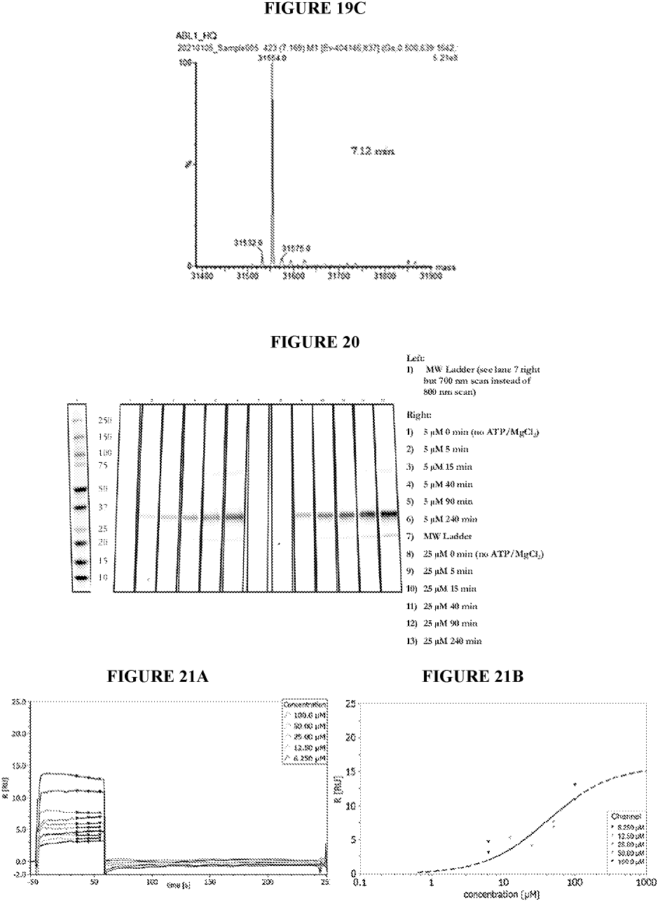

The phosphorylation state of the final protein was checked by western blot using anti-phosphor-Tyr393 Ab (Abeam ab4717). ABL1 was found completely dephosphorylated. As illustrated in FIGS. 19A-19C, this was further confirmed by LC-MS with electrospray ionization, which clearly demonstrated the protein was in the unphosphorylated state by yielding a measured molecular weight is 31,554 daltons for a calculated molecular weight of 31,555 daltons.

The activity of ABL1(T315I)(229-499) as an active kinase was demonstrated by an autophosphorylation reaction when ATP/MgCl2 was added to the purified ABL1(T315I)(229-499) and incubated (see FIG. 20). The protocol was adapted to generate phosphorylated 10xHis-tev-ABL1(T315I)(229-499) for use in the SPR studies. Briefly, ABL1 was completely dephosphorylated prior to the addition of ATP/MgC12, ATP/MgCl2 was added to initiate autophosphorylation of ABL1. The amount of phosphorylated kinase increased over time indicating that ABL1 was active. It was noted that autophosphorylation of the 25 µM sample was faster than autophosphorylation of the 5 µM sample as autophosphorylation is an intermolecular reaction and therefore was concentration dependent.

### Example XVI

### Evaluation of the Binding of Chemical Analogs to Mutant ABL1(T315I) Hinase Domain

SPR testing was performed on 21 compounds selected by the INTER net sort using the fishing net principle.

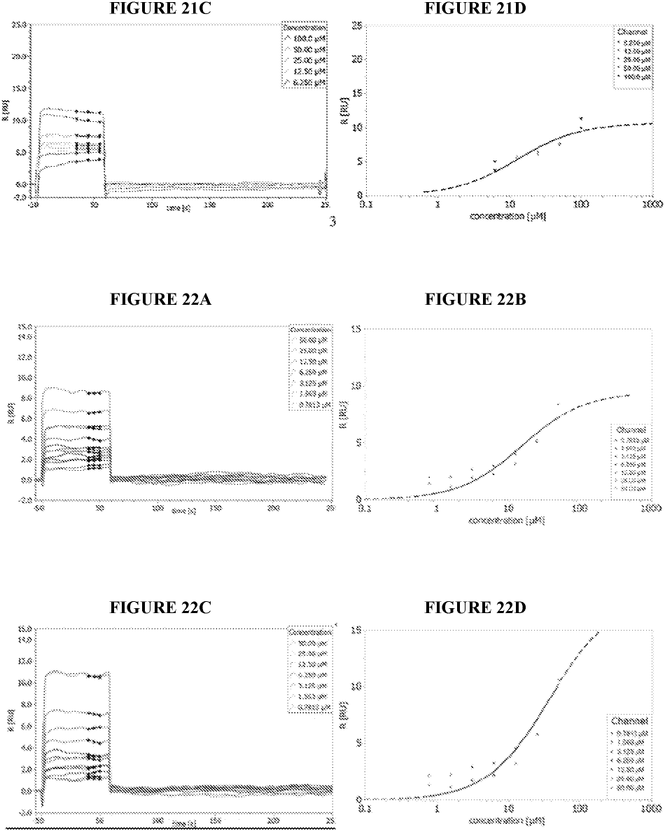

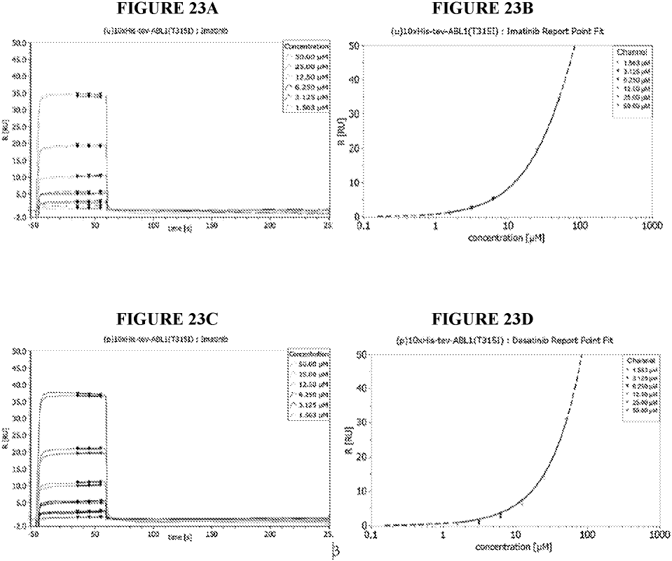

The SPR binding measurements represent the controls and the five different scaffolds from our conceptually and computationally designed molecules. The TKI inhibitors, Dasatinib and Imatinib, and a third inhibitor, DCC 2036 or Rebastinib, were used as controls for testing the chemical analogs selected based on the DFG INTER conformation virtual screening of abl T315I. The SPR measurements were performed in two different buffer systems to evaluate potential buffer effects (HEPES vs Phosphate) to develop the methods and replicate the results. Sensograms of the eSPR binding studies are presented for scaffold A-001 (FIGS. 21A-21D) and scaffold J-024 (FIGS. 22A-22D) and should be contrasted with the ineffective binding of imatinib as presented in the sensogram in FIGS. 23A-23D.

The Surface Plasmon Resonance (SPR) analysis was developed for measuring A-0001 compound binding to the unphosphorylated and to the phosphorylated forms of ABL1(229-499)(T315I).

Surface plasmon resonance required the attachment of the ligand to a surface and then flowing the analytes across this surface to measure the kinetic rate of the association (K_{A}) and the of kinetic rate dissociation (K_{D}) for a ligand interacting with the immobilized protein. From this measurement the equilibrium binding constant (K_{D}) is calculated. The SPR method was developed to enable the testing of compound binding against both the unphosphorylated and phosphorylated forms of 10xHis-tev-ABL1(T315I)(229-499).

SPR binding measurements have been performed for A-0001 and J-024. The SPR measurements were performed in two different buffer systems to evaluate potential buffer effects (HEPES vs Phosphate) to develop the methods and replicate the results. It was also confirmed that the ABL1(T315I) does not significantly bind (>100 µM) to Imatinib (approved for 1^{st} line therapy) and Dasatinib (approved for 2^{nd} line therapy), demonstrating the drug-resistant affect that the T315I mutation has (see Table 8 and FIGS. 21A-21D, FIGS. 22A-22D and FIGS. 23A-23D).

In striking contrast two more scaffolds, J and A, of the selected compounds from the INTER sort using the “fishing net” have shown steady state kinetics and low micromolar affinities against T315 I -drug resistant mutant of ABL as clinically observed in CMI, patient treated with two FDA approved drugs. Table 6 clearly illustrates that data. The virtual screen using the INTER conformation of CML ABL resulted in significantly smaller compounds than the FDA approved drugs. Those compounds will now be the subject of DNA SEQ SAR studies by medicinal chemistry aided by crystallography.

TABLE 8

Summary of current SPR results for compounds binding to the unphosphorylated or phosphorylated ABLI(T31SI)(229-499) proteins. Attachment to the biosensor surface was through Ni-NTA capture of the 1 OxHis-tag present on the N-terminus.

| Scaffold ID-# | MW (daltons) | Unphosphorylated ABL1(T315I)(229-499) in HBST buffer KD (µM) | Phosphorylated ABL1(T315I) (229-499) in HBST buffer KD (pM) | Unphosphorylated ABL1(T315I)(229-499) in PBST buffer KD (µM) | Phosphorylated ABL1(T315I)(229 -499) in PBST buffer KD (µM) |
|---|---|---|---|---|---|
| J-001 | 247 | >100 | 74 µM | >100 | >100 |
| J-002 | 326 | >100 | >100 | >100 | >100 |
| J-003 | 215 | NB | NB | NB | NB |
| J-024 | 281 | 15 µM | 38 µM | 23 µM | 22 µM |
| J-025 | 302 | 20 µM | 18 µm | 10 µM | 12 µM |
| A-001 | 251 | 2 µM | 13 µM | 5 µM | 16 µM |
| A-002 | 245 | >100 | >100 | NB | NB |
| N-003 | 177 | NB | NB | NB | NB |
| N-004 | 177 | NB | NB | NB | NB |
| N-042 | 172 | NB | NB | NB | NB |
| U-018 | 209 | NB | NB | NB | NB |
| U-019 | 180 | NB | NB | NB | NB |
| U-020 | 181 | NB | NB | NB | NB |
| U-024 | 281 | NB | NB | NB | NB |
| U-027 | 181 | NB | NB | NB | NB |
| U-036 | 211 | NB | NB | NB | NB |
| Z-003 | 215 | NB | NB | NB | NB |
| Z-023 | 453 | >100 | >100 | µM | µM |
| Dasatinib | 488 | >100 | >100 | >100 | >100 |
| Imatinib | 494 | >100 | >100 | >100 | >100 |
| DCC2036 | 553 | >100 | >100 | >100 | >100 |

The KD for Dasatinib and Imatinib were greater than 100 micromolar which is consistent with the clinical resistance observed in CML patients. The KD for a more recent version of CML inhibitor (Rebastinib) suggested that this inhibitor is ineffective against unposhorylated and phosphorylated ABL mutant T315I. Overall the two FDA approved drugs (and one inhibitor in clinical trials in CMI, indication) confirmed that the drug resistance mutation, T315I, observed in clinical patients leading to CMI, drug resistance clinical manifestation, is unambiguously related to the loss of measurable binding affinity to mutant T315I.

### Example XVII

### Evaluation of the Abl1 Inhibition of A-0001

To assess inhibitory effects of A-0001 on cancer cell resistant to tyrosine kinase inhibitor such as dasatinib or imatinib, cell-based assays using patient derived cell lines will be established. The patients will be selected for having a T315I ABL1 mutation, and a drug resistant disease.

Upon confirmation of the in vitro efficacy of A-0001, preclinical evaluations will be performed. Standard SAR assessments, structure-guided drug design, and scale ups for characterization of drug-like properties and animal studies will be implemented. The specificity of the ligands for the target kinase and related kinases will be evaluated. As is standard practice with kinase inhibitors, panels of kinases will need to be assessed to confirm the relative specificity of the key compounds. Further cell-based assays to assess and confirm target engagement with the cells and to validate the mechanism of cell death will be performed. Pharmacological parameters (ADME and DMPK), and pharmacokinetic properties will be evaluated. Animal model will be developed to assess in vivo tolerability, and efficacy.

Although the disclosure has been described with reference to the above examples, it will be understood that modifications and variations are encompassed within the spirit and scope of the disclosure. Accordingly, the disclosure is limited only by the following claims.

1. A method for obtaining the specificity profile of a therapeutic agent comprising:
a) obtaining the crystal structure of the therapeutic agent;
b) identifying a DFG phosphate conformation on a target of the therapeutic agent using an algorithmic phosphate detector; and
c) obtaining the specificity profile of the therapeutic agent using the conformation of the phosphate on the target and a pattern matching algorithm with a crystal structure library,
thereby obtaining the specificity profile of a therapeutic agent.

2. The method of claim 1, wherein the phosphate is located on an activation loop of the target.

3. The method of claim 1, wherein the phosphate is a DFG IN conformation, a DFG OUT conformation, or a DFG INTERMEDIATE conformation.

4. The method of claim 1, wherein the conformation of the phosphate indicates if the target is in an active state or in an inactive state.

5. The method of claim 3, wherein a DFG IN conformation of the phosphate indicates an active state of the target, wherein a DFG OUT conformation indicates an inactive state of the target, and wherein a DFG INTERMEDIATE conformation indicates an active state of the target.

6. The method of claim 1, wherein the target is a kinase, and the therapeutic agent is a kinase inhibitor or a drug for the treatment of cancer.

7-8. (canceled)

9. The method of claim 6, wherein the drug for the treatment of cancer is selected from the group consisting of dasatinib, nilotinib, imatinib, bosutinib, regorafenib, sorafenib, ponatinib, sunitinib, vermurafenib, vandetanib, ibrutinib, abemaciclib, ribociclib, palbociclib, axitinib, crizotinib, gilteritinib, erlotinib, midstaurin, ruxolitinib, brigatinib, and osimeritinib.

10. The method of claim 1, wherein a mutation in a gene encoding the target results in a change in the detection of the phosphate on the target.

11. The method of claim 10, wherein the mutation induces a lack of detection of a phosphate on DFG INTERMEDIATE conformation on the target.

12. (canceled)

13. The method of claim 1, wherein the crystal structure library comprises a kinase crystal structure database, a receptor crystal structure database, and/or a therapeutic agent crystal structure database.

14-15. (canceled)

16. A method of designing a scaffold of a therapeutic agent directed against a drug-resistant target comprising:
a) creating a three-dimensional fishing net of the distances in the DFG phosphate conformation (3D surface net) of the drug resistant target using an algorithmic phosphate detector;
b) screening a library of small fragment to capture small fragment that specifically binds to the 3D surface net of the target, thereby identifying a scaffold structure of the therapeutic agent; and
c) using a fragmentation algorithm to deconstruct the resistant drug structure and construct the scaffold of a therapeutic agent from the surface net structure and the captured small fragment, thereby designing the scaffold of the therapeutic agent.

17. The method of claim 16, wherein the target is a kinase, and the therapeutic agent is a kinase inhibitor.

18. The method of claim 16, wherein a mutation in a gene encoding the target results in a change in the conformation of the phosphate on the target.

19. The method of claim 18, wherein the mutation induces a phosphate to be in a DFG INTERMEDIATE conformation.

20. The method of claim 16, wherein the drug-resistant target is in a DFG INTERMEDIATE conformation, and wherein the 3D surface net is a 3D INTERMEDIATE surface net (3D INTER net).

21. The method of claim 17, wherein creating the three-dimensional fishing net comprises excluding regions of high frequency of drug resistance mutation of the kinase.

22. The method of claim 21, wherein regions of high frequency of drug resistance mutation of the kinase comprises HVR regions 1-7.

23-25. (canceled)

26. The method of claim 16, wherein the scaffold of the therapeutic agent is defined by two derivation constituents constituents left open to generate a combinatorial array of analogs.

27-29. (canceled)

30. The method of claim 16, wherein the target is kinase ABL1 mutant T315I.

31. The method of claim 30, wherein the scaffold of the therapeutic agent is selected from 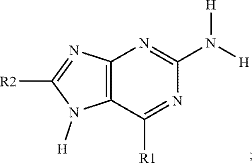 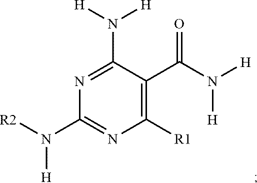 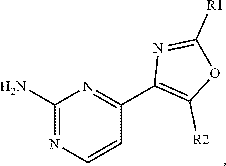 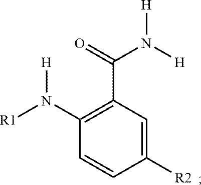 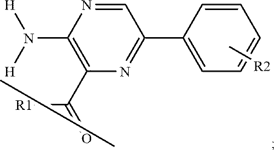 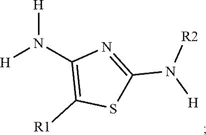 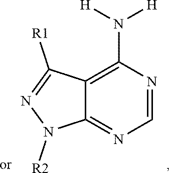
wherein R1 and R2 are two derivation constituents.

32. A method of treating cancer in a subject comprising administering to the subject a therapeutically effective amount of a therapeutic agent,
wherein the cancer is characterized by a mutation in an ABL1 gene, and wherein the therapeutic agent is selected from 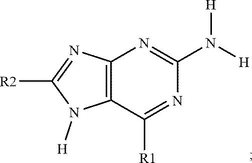 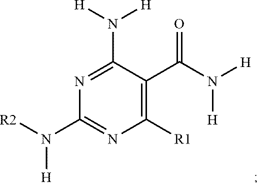 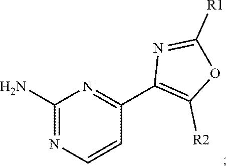 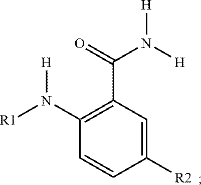 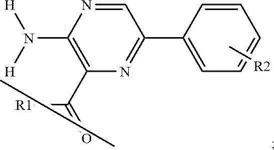 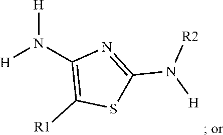 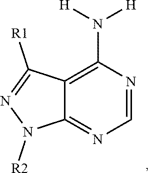
wherein R1 and R2 are two derivation constituents, thereby treating cancer in the subject.

33-35. (canceled)

36. The method of claim 32 , wherein the therapeutic agent is CDK inhibitor NU6027.

37-41. (canceled)

42. The method of claim 32, wherein the cancer is a tyrosine kinase inhibitor (TKI) resistant leukemia is selected from the group consisting of acute lymphoblastic leukemia (ALL), acute myeloid leukemia (AML), blastic plasmacytoid dendritic cell neoplasm (BPDCN), chronic lymphocytic leukemia (CLL), chronic myelogenous leukemia (CML), hairy cell leukemia, mast cell leukemia and meningeal leukemia.

43-47. (canceled)
